(19) United States
(12) Patent Application Publication
(10) Pub. No.: US 2025/0166762 A1
(43) Pub. Date: May 22, 2025

(54) CLINICAL WORKFLOWS UTILIZING PATIENT REPORT SUMMARIZATION AND Q&A TECHNOLOGIES

(71) Applicant: GE Precision Healthcare LLC, Waukesha, WI (US)

(72) Inventors: Omer Barkol, Haifa (IL); Michail Fanariotis, Skien (NO); Farid Ghanayim, Baqa al-Gharbiyye (IL); Reuth Goldstein, Haifa (IL); Renato Keshet, Haifa (IL); Zoltán Kiricsi, Budapest (HU); Praveen Kumar Suresh, Salem (IN); Annamraju Ravi Bhardwaj, Bengaluru (IN)

(21) Appl. No.: 18/930,532

(22) Filed: Oct. 29, 2024

(30) Foreign Application Priority Data
Nov. 21, 2023 (IN) 202341079185

(51) Int. Cl. G16H 15/00; G16H 30/20

(52) U.S. Cl. CPC G16H 15/00; G16H 30/20

(57) ABSTRACT

The described technology is generally directed towards automated and dynamic creation of a consolidated summary of medical information pertaining to a patient. Rather than a caregiver having to trawl through numerous reports and medical information, utilizing consolidated reports enables efficient review of medical information to enable expedited determination and treatment of the patient. In an example sequence of operation, a collection of patient/medical reports can be individually summarized to form a collection of summarized versions of the original reports. The summarized reports can be further collectively summarized to generate a consolidated summary. Hence, the consolidated summary is a summary of the initial collection of patient/medical records. A consolidated summary can comprise patient information, medical history, medical images, pertinent medical research, etc. A consolidated summary can be presented on an interactive display, with the respective initial patient/medical reports accessible via selectable hyperlinks presented in the consolidated summary.

### CROSS-REFERENCE TO RELATED APPLICATIONS

This application claims priority to India Provisional Patent Application No. 202341079185 filed on Nov. 21, 2023, entitled “CLINICAL WORKFLOWS UTILIZING PATIENT REPORT SUMMARIZATION AND Q&A TECHNOLOGIES”. The entireties of the aforementioned application are incorporated by reference herein.

### TECHNICAL FIELD

This application relates to systems and techniques facilitating analysis and presentation of medical information regarding a patient.

### BACKGROUND

Technological advancements in recent years have prompted the medical world to strive for precision healthcare. The goal of precision healthcare comprises the ability for a caregiver to retrieve precise information (about the patient and/or about treatment) at the right time and place. A consideration driving precision healthcare relates to the time available to review a patient's medical data, whereby the available time tends to become shorter as the workload and working requirements of medical staff increases (e.g., as radiologists and clinicians work intensifies). Another consideration is the ability for prompt and simple querying of information sources.

### SUMMARY

The following presents a simplified summary of the disclosed subject matter to provide a basic understanding of one or more of the various embodiments described herein. This summary is not an extensive overview of the various embodiments. It is intended neither to identify key or critical elements of the various embodiments nor to delineate the scope of the various embodiments. The sole purpose of the Summary is to present some concepts of the disclosure in a streamlined form as a prelude to the more detailed description that is presented later.

In one or more embodiments described herein, systems, devices, computer-implemented methods, configurations, apparatus, and/or computer program products are presented to automatically and dynamically generate one or more consolidated summaries comprising, and linked to, summary reports and further to patient reports, in accordance with an embodiment.

According to one or more embodiments, a system is presented, comprising at least one processor and at least one memory coupled to the at least one processor and having instructions stored thereon, wherein, in response to the at least one processor executing the instructions, the instructions facilitate performance of operations, comprising generating a first report summary in a collection of report summaries, wherein the first report summary comprises a first summary of first patient medical data in a first patient report pertaining to a patient, and the first report summary is generated in accordance with a first prompt applied to a large language model operating on the first patient report. In a further embodiment, the operations can further comprise generating a second report summary in the collection of report summaries, wherein the second report summary comprises a first summary of second patient medical data in a second patient report pertaining to the patient, and the second report summary is generated in accordance with the first prompt applied to the large language model operating on the first patient report. In a further embodiment, the operations can further comprise generating a consolidated summary of a patient's medical history pertaining to the patient, wherein the consolidated summary is generated from the first report summary and the second report summary, and the consolidated report is generated in accordance with a second prompt applied to the large language model operating on the first report summary and the second report summary. In a further embodiment, the operations can further comprise presenting the consolidated summary for review.

In a further embodiment, the operations can further comprise identifying first content in the consolidated summary pertaining to second content in a query received at the system, wherein identification of the first content is based on vector similarity between the first content in the consolidated summary and the second content of the query. In a further embodiment, the operations can further comprise presenting the first content for review.

In a further embodiment, the operations can further comprise detecting a selection from the consolidated summary, further determining patient medical data corresponding to the selection, and further presenting the patient medical data pertaining to the selection.

In another embodiment, the consolidated summary can be presented on a human-machine interface (HMI), and at least one of the first patient medical data pertaining to the selection is co-presented with the consolidated summary on the HMI or the second patient medical data pertaining to the selection is co-presented with the consolidated summary on the HMI.

In a further embodiment, wherein the first patient medical data and the second patient medical data comprises at least one of an image, medical information pertaining to the patient, patient personal information, or medical research information pertinent to the patient.

In an embodiment, the at least one medical report can comprise at least one of a radiology report, a oncology report, a cardiology report, a neurology report, a patient intervention report, an intensive care report, a general practitioner report, a report pertaining to the patient, a note pertaining to the patient, a report in an electronic medical record (EMR) system pertaining to the patient, a report in a non-EMR system pertaining to the patient, or a medical specialty report.

In another embodiment, a first report summary can comprise a summary of patient data included in at least one medical report. In a further embodiment, the first report summary can comprise a summary of at least one of patient activity or patient testing performed between a first date and a second date.

In another embodiment, the consolidated summary can further include one or more links, wherein a first link is a hyperlink to a first medical report in the at least one medical report, and the operations can further comprise: detecting selection of the first link, and further presenting the first medical report.

In a further embodiment, the operations can further comprise parsing the query to identify a subject of interest, further identifying content in the consolidated summary pertaining to the subject of interest, further identifying a first report pertaining to the first content in the consolidated summary, wherein the first report was summarized to create the consolidated summary, and further presenting, in response to the query, the first report.

In another embodiment, wherein the at least one medical report comprises a plurality of medical reports, and the consolidated summary is a first consolidated summary comprising oncology data generated from a first report summary, wherein the first report summary is generated from one or more oncology reports included in the plurality of medical reports, and a second consolidated summary comprises radiology data generated from a third report summary, wherein the third report summary is generated from one or more radiology reports included in the plurality of medical reports.

In further embodiments, a computer-implemented method is provided, wherein the method comprises creating, by a device comprising at least one processor, a first medical data summary, wherein the first medical data summary is a summary of first medical data in a first medical report relating to a patient, and further creating, by the device, a second medical data summary, wherein the second medical data summary is a summary of second medical data in a second medical report relating to the patient. In a further embodiment, the computer-implemented method can further comprise generating, by the device, a consolidated summary, wherein the consolidated summary comprises a summary of the first medical data summary and the second medical data summary, and further presenting, by the device, the consolidated summary

In a further embodiment, the computer-implemented method can further comprise generating, by the device, in the consolidated summary a hyperlink, wherein the hyperlink is a digital reference to the first medical data summary. In a further embodiment, the computer-implemented method can further comprise detecting, by the device, selection of the hyperlink, and further presenting, by the device, the first medical data summary in conjunction with the consolidated summary.

In another embodiment, the computer-implemented method can further comprise generating, by the device, in the consolidated summary a first hyperlink, wherein the first hyperlink is a first digital reference to the first medical data summary, and further generating, by the device, in the consolidated summary a second hyperlink, wherein the second hyperlink is a second digital reference to the second medical data summary. In a further embodiment, the computer-implemented method can further comprise detecting, by the device, selection of the of the first hyperlink or the second hyperlink, and (a) in response to detecting selection of the first hyperlink, presenting, by the device, the first medical data summary, and (b) in response to detecting selection of the second hyperlink, presenting, by the device, the second medical data summary.

In a further embodiment, the computer-implemented method can further comprise receiving, by the device, an input comprising a question regarding information presented in the consolidated summary, further parsing, by the device, the information in the consolidated summary to generate an answer to the question, and further presenting, by the device, the answer. In another embodiment, the computer-implemented method can further comprise prior to parsing the information in the consolidated summary, applying a first prompt to the question, wherein the first prompt applies a filter to the question to further refine the question for application in parsing the information in the consolidated summary. In another embodiment, the computer-implemented method can further comprise capturing, by the device, interaction with the answer, and further generating, by the device, a second prompt, wherein the second prompt comprises the first prompt adjusted in accordance with the interaction.

Further embodiments can include a computer program product stored on a non-transitory computer-readable medium and comprising machine-executable instructions, wherein in response to being executed, the machine-executable instructions cause a system to perform operations, comprising presenting a consolidated report, wherein the consolidated report comprises a link to a summary report, wherein the summary report is a summary of first content in a first patient report and second content in a second patient report, wherein the first patient report and the second patient report pertain to the same patient, wherein the consolidated report can be generated by a computer-based language model configured to identify third content in the summary report pertaining to a medical condition of the patient associated with the first patient report and the second patient report. In a further embodiment, the operations can further comprise, in response to detecting selection of the link, presenting the summary report.

In another embodiment, the link to the first summary report is a first link, and the first summary report further includes a second link to the first patient report and a third link to the second patient report. In a further embodiment, the operations can further comprise: (a) in response to detecting selection of the second link, presenting the first patient report, and (b) in response to detecting selection of the third link, presenting the second patient report.

In a further embodiment, the operations can further comprise receiving a query having fourth content, wherein the query pertains to fifth content presented in the consolidated report, wherein the fifth content is vectorized content. In another embodiment, the operations can further comprise vectorizing the fourth content of the query, further identifying the fifth content based on vector similarity between the vectorized fifth content and the vectorized fourth content, and further presenting the fifth content.

In a further embodiment, the computer-based language model is a first computer-based model and the consolidated report is a first consolidated report, wherein the operations can further comprise receiving a modification to the fifth content, and further updating the consolidated report in accordance with the modification. The operations can further comprise generating a second computer-based language model, wherein the second computer-based language model is generated in accordance with the modification to the consolidated report. The operations can further comprise subsequently applying the second computer-based language model to the summary report to generate a second consolidated report.

### DESCRIPTION OF THE DRAWINGS

One or more embodiments are described below in the Detailed Description section with reference to the following drawings.

FIG. 1A illustrates a system that can be utilized to dynamically summarize patient reports in conjunction with enabling efficient interaction with the reports, summaries, and suchlike, according to at least one embodiment.

FIG. 1B presents a schematic of report summaries being generated from various initial reports, and a consolidated summary (a.k.a. summary of the summaries) being generated from the report summaries, according to at least one embodiment.

FIG. 1C presents a schematic of report summaries being generated from various initial reports, and a consolidated summary being generated from the report summaries, according to at least one embodiment.

FIG. 1D presents a schematic of reports being generated over the course of a series of visits, and a summary report being generated to summarize activity, patient update, etc., for the respective dates of visits and intervening actions, according to at least one embodiment

FIG. 2 presents a screen illustrates an example screen that may be presented by a radiology application, in accordance with an embodiment.

FIG. 3 presents a screen illustrating an example screen that may be presented by a radiology application, in accordance with an embodiment.

FIG. 4 presents a screen 400 illustrating an example screen that may be presented by an oncology application, in accordance with an embodiment.

FIG. 5 presents a screen illustrating an example screen that may be presented by an oncology application, in accordance with an embodiment.

FIG. 6 illustrates a computer-implemented method for generating a summary page from a patient report, in accordance with an embodiment.

FIG. 7 illustrates a computer-implemented method for accessing further data associated with a summary page, in accordance with one or more embodiments.

FIG. 8 illustrates a computer-implemented method for applying a prompt to a question to constrain scope of question and/or supplied answer, in accordance with an embodiment.

FIG. 9 presents a schematic of a RAG process flow as can be implemented as part of a Q&A process, in accordance with an embodiment.

FIG. 10 presents a schematic illustrating respective operational flow during implementation of a report interaction system, in accordance with an embodiment.

FIG. 11 presents a flowchart of an example computer-implemented method for automatically and dynamically generating one or more consolidated summaries comprising and linked to summary reports and further to patient reports, in accordance with an embodiment.

FIG. 12 presents a flowchart of an example computer-implemented method for automatically and dynamically generating one or more consolidated summaries comprising and linked to summary reports and further to patient reports, in accordance with an embodiment.

FIG. 13 presents a flowchart of an example computer-implemented method for automatically and dynamically generating one or more consolidated summaries comprising and linked to summary reports and further to patient reports, in accordance with an embodiment.

FIG. 14 is a block diagram illustrating an example computing environment in which the various embodiments described herein can be implemented.

FIG. 15 is a block diagram illustrating an example computing environment with which the disclosed subject matter can interact, in accordance with an embodiment.

### DETAILED DESCRIPTION

The following detailed description is merely illustrative and is not intended to limit embodiments and/or application or uses of embodiments. Furthermore, there is no intention to be bound by any expressed and/or implied information presented in any of the preceding Background section, in the Detailed Description section, and/or in the Abstract.

One or more embodiments are now described with reference to the drawings, wherein like referenced numerals are used to refer to like elements throughout. In the following description, for purposes of explanation, numerous specific details are set forth in order to provide a more thorough understanding of the one or more embodiments. It is evident, however, in various cases, that the one or more embodiments can be practiced without these specific details.

It is to be understood that when an element is referred to as being “coupled” to another element, it can describe one or more different types of coupling including, but not limited to, chemical coupling, communicative coupling, electrical coupling, electromagnetic coupling, operative coupling, optical coupling, physical coupling, thermal coupling, and/or another type of coupling. Likewise, it is to be understood that when an element is referred to as being “connected” to another element, it can describe one or more different types of connecting including, but not limited to, electrical connecting, electromagnetic connecting, operative connecting, optical connecting, physical connecting, thermal connecting, and/or another type of connecting.

As used herein, “data” can comprise metadata. Further, ranges A-n are utilized herein to indicate a respective plurality of devices, components, signals etc., where n is any positive integer.

### OVERVIEW

During a patient's lifetime, their medical history may become quite complex and voluminous with numerous reports, images, etc., generated and compiled throughout the lifetime. It can become a complicated task when a medical professional is interested in reviewing/assessing one or more aspects of the patient's medical condition. For example, a wealth of data and reports can be acquired regarding a patent who is undergoing/has undergone cancer treatment. Hence, when an oncologist is to review/analyze the reports to identify aspects of the patient's medical history pertaining to a current condition of concern/interest, the volume of patient information can preclude expedited and accurate diagnosis or clinical assessment.

Per the various embodiments presented herein, rather than presenting a plethora of information regarding a patient's medical condition and accordingly, requiring a medical expert to navigate through the wealth of data, advantage can be taken of technologies, such as artificial intelligence (AI) and machine learning (ML) technologies, to identify/determine insights (aka findings, potential diagnoses, issues of concern, and suchlike), summarise the information, and further, present the information as one or more summary screens presented on a summary interface, whereby a caregiver can interact with the one or more summary screens to navigate through respective reports and summaries as part of the condition diagnosis.

In the various embodiments presented herein, various technologies and processes can be applied to:
(a) summarize each medical report pertaining to the patient;
(b) process the respective summaries of the respective patient reports to generate a consolidated summary, wherein the consolidated summary can be considered to be a “patient summary”. Processing of the respective summaries can include any suitable/applicable operation(s), such as identifying pertinent information, extracting information, consolidating information, and the like;
(c) the consolidated summary can be presented (e.g., on an interactive display) in conjunction with one or more links (a.k.a. hyperlinks, e.g., a digital reference to data, presented with first information/data that when clicked, presents second information/data).
   (i) the one or more links can respectively link directly to a full version of a report or a report page, e.g., without presenting a summary as an interim step (e.g., per (c)(ii) below). When a link is selected (e.g., by clicking with a mouse, hovering a cursor over, selection via a touchscreen, and suchlike) the report associated with the link can be presented, e.g., as a pop-up window on the screen. In a further embodiment, a particular sentence in the presented report can be highlighted, wherein the sentence is a specific source of information conveyed in a link.
   (ii) the one or more links can respectively link to/represent each of the individual report summaries/patient reports. In an example scenario, a consolidated summary presented on a screen can be a summary of thirty reports regarding a patient's cancer treatment. Throughout the consolidated summary, various links are presented regarding findings, etc., which when selected (e.g., by clicking with a mouse, hovering a cursor over, and suchlike), a summary of the report associated with the link can be presented, e.g., as a pop-up window on the screen, and when further selected, the link can cause a full version of the summarized report to be presented;

d) enable interaction with the summaries and/or reports via keyboard, voice interaction, etc., between a medic and the report system. Various embodiments are presented herein regarding question and answer (Q&A) operations. In an embodiment, a retrieval-augmented generation (RAG) process can be utilized, whereby a vectorized index of sentences in a patient report enables vector/similarity operations to be performed between content/context of a question and content of a patient report, enabling a pertinent answer to be derived. In another embodiment, a collection of report summaries can be compiled and accessed as a source for the question-answering process, which can use any suitable technology, e.g., vector/similarity.

In an aspect, the consolidated summary can comprise, in a non-limiting list, any of:
a) a summary of all of medical reports for a patient,
b) a summary of changes between a first report and a second report, or
c) a summary of what happened between a first date and a second date regarding, for example, a patient's condition, treatment, and suchlike.

Hence, by creating and implementing (i) report summaries and (ii) a consolidated summary and/or (iii) a vectorized index, a potentially cumbersome amount of data can be effectively reduced to a lesser volume of data, enabling accurate and expeditious interaction with the consolidated summary, the underlying report summaries, and further, the full reports from which the report summaries and consolidated summaries were generated.

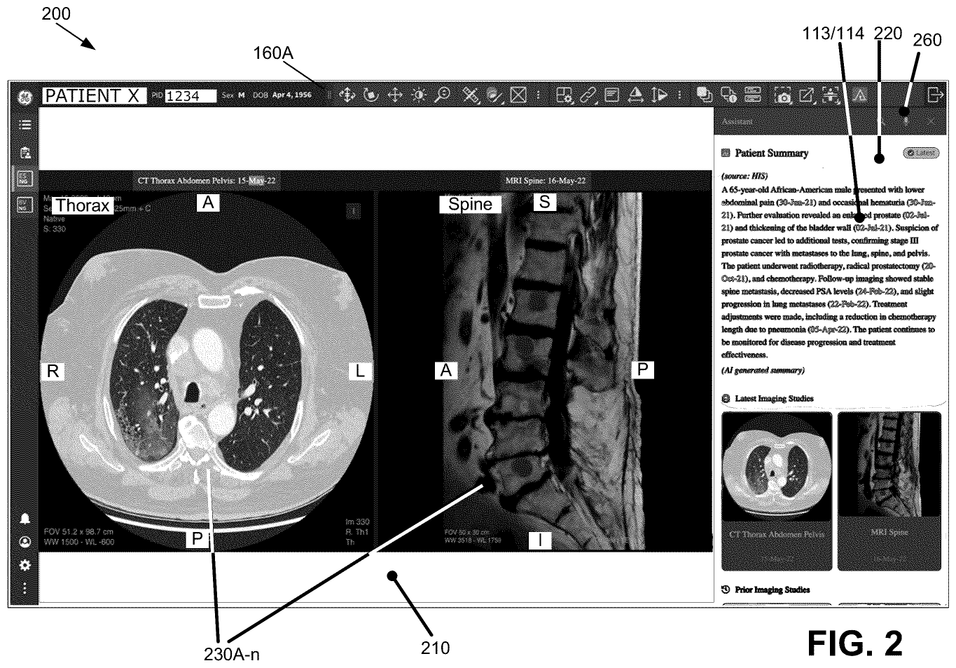

It is to be appreciated that while the various embodiments presented herein relate to presentation of a consolidated summary being presented on a screen (e.g., per FIG. 2), more than one consolidated summary can be concurrently/co-presented on the same screen. For example, a first consolidated summary regarding a patient's oncology information can be co-presented with a second consolidated summary regarding the patient's radiology information.

Further, prompt-based technologies can be utilized to enable interaction with the reports, summaries, etc., wherein the prompts provide necessary input to an AI model (e.g., a large language model (LLM)) to instruct the model with a required task (e.g., compute a report summary, generate a patient summary, answer a question, and the like). Prompt-based technologies can further function to constrain/filter identification and presentation of the reports, summaries, etc., enabling focused interaction between a caregiver and the reports/summaries.

An advantage of the one or more systems, computer-implemented methods and/or computer program products presented herein is enabling presentation of a summary of patient data to enable a caregiver to expeditiously access patient information to enable efficient and quick diagnosis of the patient's condition, while minimizing the amount of time the medic has to engage with the data to make the diagnosis, decision, etc. Accordingly, advantage can be taken of AI/ML technology to parse/summarize the patient data and limit an initial presentation of patient data to information, findings, etc., that are/may be pertinent to the diagnosis, wherein the initial presentation of patient data can comprise of one or more findings, which upon initial or subsequent review can be utilized to assist with determination of one or more diagnoses.

It is to be appreciated that while the various embodiments presented herein are directed towards application of AI/ML to summarizing and presenting medical data, the embodiments are equally applicable to any comparable use, e.g., presentation of veterinary information, presentation of financial data, failure diagnostics, and suchlike. Further, the various embodiments are applicable to any data form, e.g., structured data such as measurements, vitals, data entered in a systematic manner in accordance with prompts, textboxes, and the like, and unstructured data such as physicians notes, an entry provided by a caregiver in accordance with a textbox, document scan, and the like.

### Consolidated Summary System

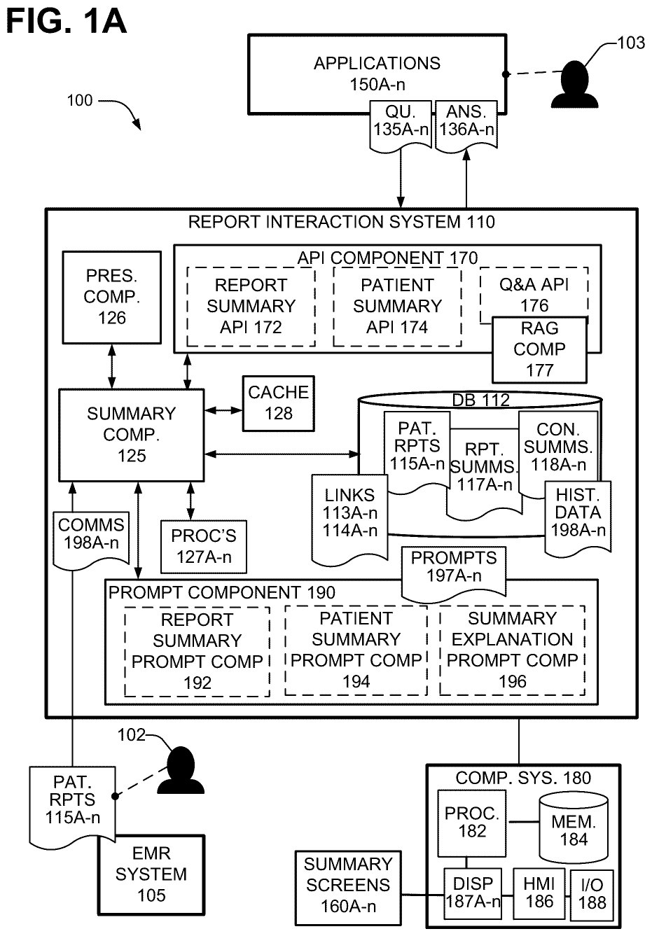

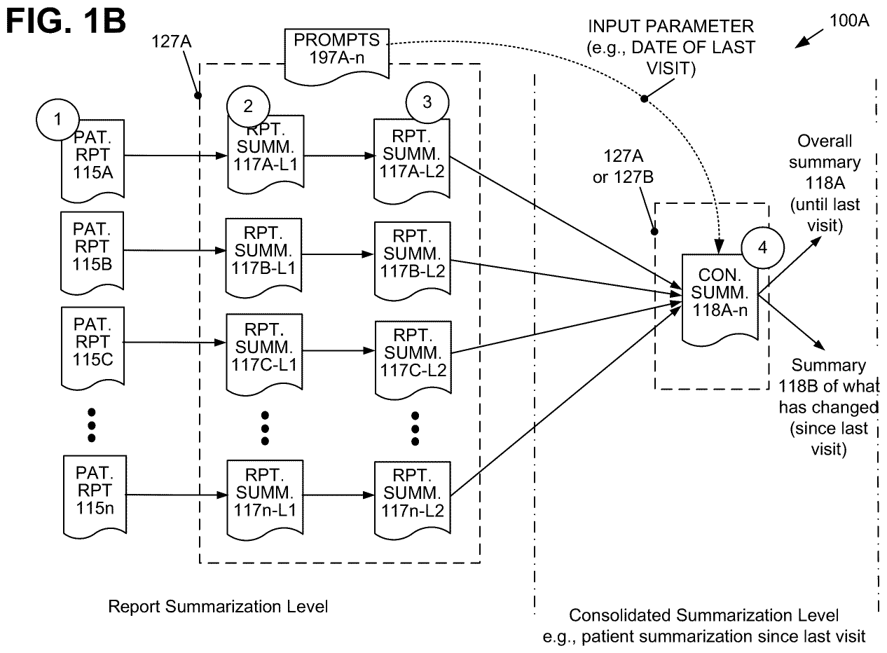

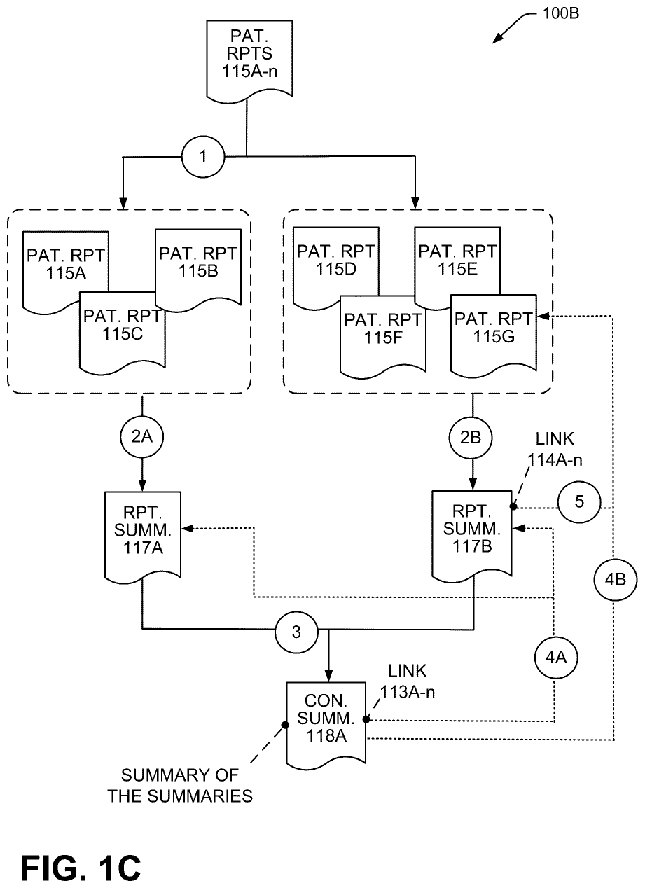

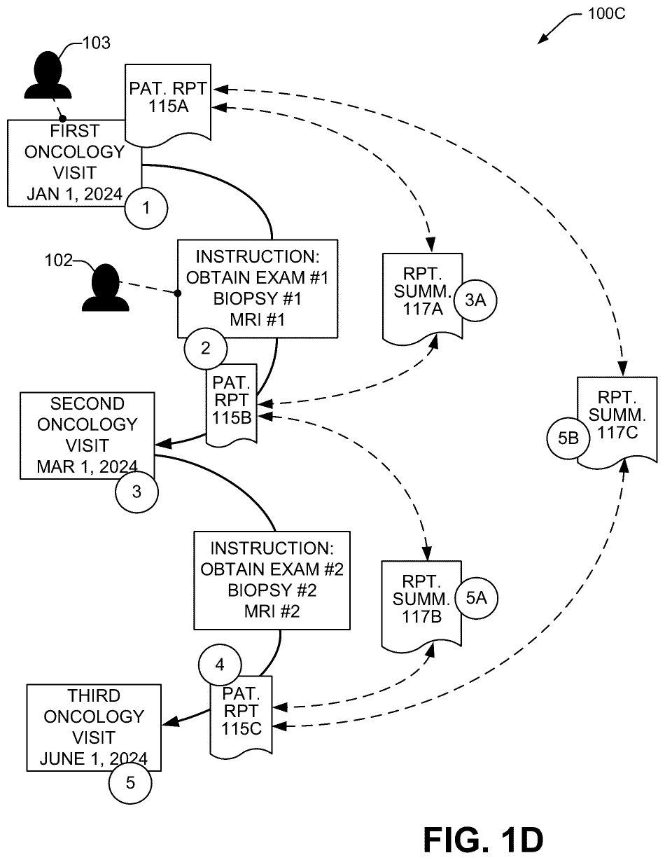

Turning now to the drawings, FIG. 1A illustrates a system 100 that can be utilized to dynamically summarize patient reports in conjunction with enabling efficient interaction with the reports, summaries, and suchlike, according to at least one embodiment.

System 100 comprises a report interaction system (RIS) 110 configured to (a) generate respective report summaries 117A-n from patient reports 115A-n, (b) further generate a consolidated summary 118A-n (a.k.a. a “summary of the summaries”, a patient summary) from the report summaries 117A-n, and (c) further enable interaction with the consolidated summary 118A-n to enable a caregiver 103 to quickly access a patient's medical information and have confidence in the veracity and integrity of the information (e.g., reports and summaries) presented/available to the caregiver 103. The term caregiver 103 is used herein to represent any person involved in reviewing medical/patient data, such as a doctor, nurse, radiologist, medical specialist, medical professional, and suchlike.

In an embodiment, applications 150A-n form a front-end of system 100, with access to the back end RIS 110 via respective application programming interface(s) (API's), e.g., a report summary API 172, a patient summary API 174, and/or a Q&A API 176.

Patient reports 115A-n, report summaries 117A-n, and consolidated summary 118A-n can comprise text, alphanumerics, numbers, single words, phrases, short statements, long statements, expressions, context, tables, JSON objects, etc. Reports 115A-n and summaries 117A-n and 118A-n can be of any suitable format/filetype, such as a PDF, DICOM PDF, word processor document, spreadsheet, and suchlike, and can further include medical condition data regarding the patient 102, medical history, medication history, vital signs, lab exam results, radiology and pathological results, medical consult assessments, structured data, unstructured data, and suchlike, and further include any images associated with treatment of patient 102, e.g., magnetic resonance imaging (MRI), X-ray image, mammogram image, scans, and suchlike.

As shown, patient reports 115A-n (and other data, images, metadata, and suchlike) can be generated by an electronic medical record (EMR) system 105, or any system configured to stream, store, and/or generate information, data, etc., regarding a patient 102's condition. Reports 115A-n can be received at RIS 110 (e.g., via an I/O 188, as further described) and stored in a database 112 (e.g., in memory 184, as further described).

As further shown, reports 115A-n can be received at/applied to a summary component 125, wherein the summary component 125 can be configured to generate a respective report summary 117A-n for each of the received reports 115A-n, such that, for example, n summaries can be generated for n reports. Summary component 125 can be further utilized to generate a consolidated summary 118A-n from the collection of report summaries 117A-n. The summary reports 117A-n and/or the consolidated summaries 118A-n can comprise the summaries only (e.g., of patient reports 115A-n/summary reports 117A-n), or also additional information/data/metadata, such as the report date, report type, etc., associated with each summary. Summary component 125 can utilize any suitable format such as free text, a table(s), a JSON object, or other applicable representation.

In an embodiment, summary component 125 can utilize one or more processes 127A-n, wherein processes 127A-n can comprise various AI and ML technologies configured to summarize text, sentences, report data, and suchlike, in reports 115A-n and/or summaries 117A-n and 118A-n. As further described, any suitable/applicable AI/ML technologies can be utilized by summary component 125, for example, process 127A can comprise a large language model (LLM) configured to analyze the natural language format of the reports 115A-n to generate the report summaries 117A-n, and further apply LLM process 127A to the report summaries 117A-n to analyze the content of the report summaries 117A-n to further compile/generate the consolidated summary 118A. Report summaries 117A-n and consolidated summaries 118A-n can be stored in database 112. Database 112 can further include historical data 198A-n comprising prior reports, summaries, etc., which can be utilized to train processes 127A-n to accurately identify and summarize information in the reports 115A-n and summaries 117A-n/118A-n. It is to be appreciated that the processes 127A-n can be utilized by any of the components included in RIS 110, such as summary component 125, presentation component 126, API component 170, RAG component 177, prompt component 190, and suchlike. In an embodiment, database 112 can be included in memory 184.

In an embodiment, as reports 115A-n are received at RIS 110, summary component 125 can be configured to generate a report summary 117A-n for the newly received reports and further store the respective report summary 117A-n in database 112/memory 184. Hence, as report 115R is received, report summary 117R is generated and stored, as report 115S is received, report summary 117S is generated and stored, etc.

As further described (e.g., as presented in FIGS. 1B and 1C), during creation of a consolidated summary 118A, the summary component 125 can be configured to generate links/hyperlinks 113A-n (aka first links) linking the consolidated summary 118A to each of the report summaries 117A-n utilized by summary component 125 to create consolidated summary 118A. Further, the summary component 125 can be further configured to generate links/hyperlinks 114A-n (aka second links) linking a report summary 117A-n to the reports 115A-n used by the summary component 125 to create the respective report summary 117A-n. Alternatively, the consolidated summary 118A-n may be linked directly to the corresponding reports 115A-n. Hyperlinks 113A-n and 114A-n can be stored in the database 112 associated with the respective reports and summaries 115A-n, 117A-n, and 118A-n to which the hyperlinks 113A-n and 114A-n pertain and connect. Links 113A-n and 114A-n can be utilized to navigate (e.g., by selecting) between reports 115A-n, report summaries 117A-n, and consolidated summaries 118A-n.

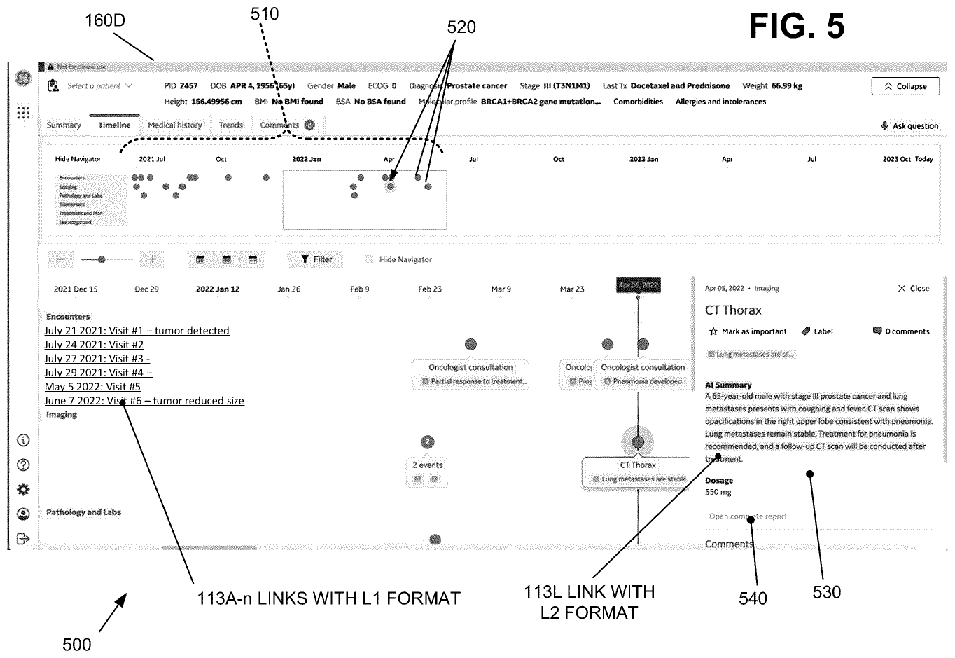

In an embodiment, the amount of information presented in any of a consolidated summary 118A-n, a report summary 117A-n, a link 113A-n or 114A-n, etc., can be of any amount. For example, a consolidated summary 118A-n, link 113A-n/114A-n, etc., can comprise a short statement/heading (e.g., 10 words maximum) being presented as a summary (e.g., per FIG. 5), referred to herein as an L1 level format. Alternatively, a consolidated summary 118A-n, a link 113A-n/114A-n, etc., can be presented with a L2 level format, wherein a L2 level format can be a format where a greater number (e.g., more than 10 words, between 10 and 150 words, a maximum of 200 words, and the like) of words are presented. In another example scenario, a first link (e.g., first link 113A, first link 114A) and a second link (e.g., second link 113A, second link 114A) can link to the same report (e.g., a report summary 117A, a patient report 115A), whereby, the report is listed in a timeline of treatments/documents and the first link is presented with an L1 short form format, and further, a summary of the report (e.g., as a report summary 117A) can be presented with the second link being an L2 long form format.

RIS 110 can further include a presentation component 126 configured to control presentation on any of reports 115A-n, report summaries 117A-n, consolidated summaries 118A-n, links 113A-n/114A-n, historical data 198A-n, questions 135A-n, answers 136A-n, prompts 197A-n, and suchlike on summary screens 160A-n (e.g., per FIGS. 2-5) on a display (e.g., display/screens 187A-n). In an embodiment, presentation component 126 can be configured to control which information to present, e.g., images pertaining to a summary 117A-n/118A-n, the underlying patient report 115A-n, timelines of reports 115A-n being generated, summaries 117A-n/118A-n or portions thereof, links 113A-n/114A-n and information to be accessed by the links 113A-n/114A-n, and suchlike. Summary screens 160A-n can comprise of various portions, e.g., for image display, summary presentation, and suchlike, as well as pop-up screens. In an embodiment, the applications 150A-n can provide/generate/configure summary screens (e.g., summary screens 160A-n local to an application 150A-n) on which any of reports 115A-n, report summaries 117A-n, consolidated summaries 118A-n, etc., can be presented. Hence, a third party owner of the application 150A-n can configure their applications/screens in accordance with their needs while interfacing with RIS 110 (e.g., via APIs provided by API 170). In a further embodiment, RIS 110 can provide widgets (e.g., display packages) which developers of applications 150A-n can incorporate into their respective applications with RIS 110 configured to provide the any of reports 115A-n, report summaries 117A-n, consolidated summaries 118A-n, links 113A-n/114A-n, historical data 198A-n, and further respond with answers 136A-n (a.k.a. a RAG-based answer) to questions 135A-n provided by the third party caregiver 103 at the local application 150A-n.

Conventional systems present a totality of information available in reports 115A-n, however, the volume of data can render the task of medical condition review/diagnosis by a caregiver 103 to be time consuming and also potentially fraught with misdiagnosis given the wealth of data to review. However, by utilizing the one or more embodiments presented herein regarding summarizing content of patient reports 115A-n as report summaries 117A-n and/or consolidated summaries 118A-n enables caregiver 103 to quickly, accurately, and with confidence, make a determination of whether a medical condition exists or not.

A variety of software applications 150A-n can be communicatively coupled to/interact with RIS 110. Per the various example scenarios presented herein pertaining to medical reports and data, applications 150A-n can include various medical specialties such as radiology, oncology, pathology, cardiology, neurology, patient intervention, intensive care, general practitioner, and suchlike, and further, a patient report 115A-n can be an outpatient note, an inpatient note, emergency department report, a note pertaining to patient 102, a report pertaining to patient 102, and suchlike. It is to be appreciated that while the term report is used herein, any suitable content/object can be used, e.g., a note, a scanned document, a verbal recording, and suchlike. The patient report 115A-n can be included in the EMR system 105. The patient report 115A-n can also be a non-EMR document, for example, when a patient 102 is being transferred from a first hospital (having a first EMR system 105A) to a second hospital (having a second EMR system 105B) and the patient report 115A in EMR system 105A has still to be entered in the second EMR system 105B, such that the patient 102 is accompanied with a physical, printed copy of patient report 115A.

However, it is to be appreciated that while the various embodiments presented herein are directed towards healthcare, the various embodiments can be directed towards any suitable implementation, such as transport, finance, manufacturing, education, and suchlike. Applications 150A-n can be configured to enable an entity, e.g., a caregiver 103, to access and review any of reports 115A-n, report summaries 117A-n, and/or consolidated summaries 118A-n, e.g., as part of medical review and treatment of patient 102, and further conduct Q&A interaction.

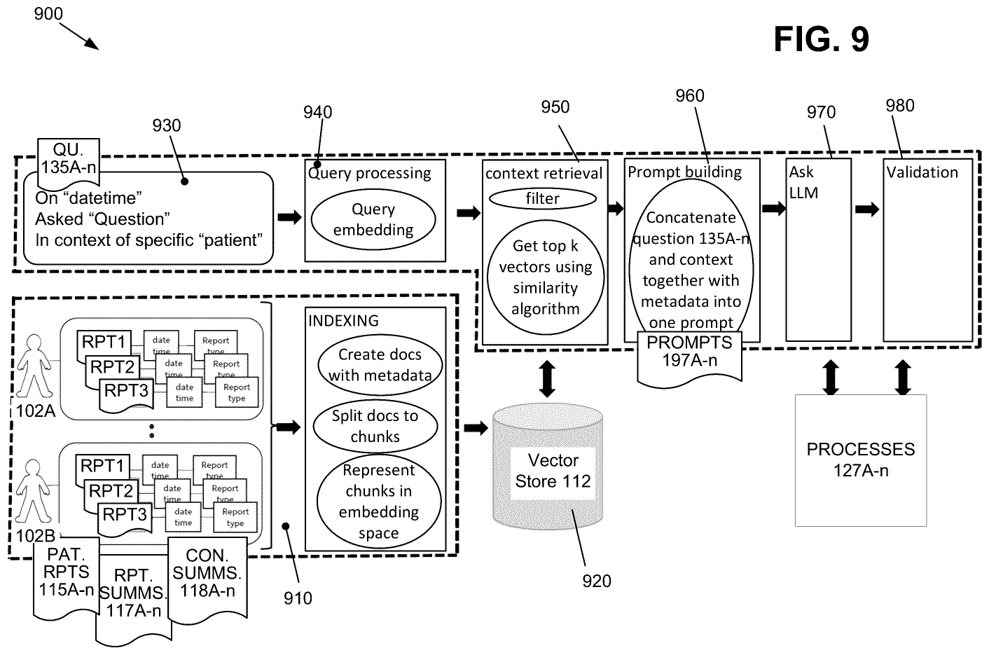

Interaction of the applications 150A-n with respective components of RIS 110 can be via one or more APIs, as controlled by API component 170. A report summary API 172 can be utilized to enable interaction between an application 150A-n with any of reports 115A-n and report summaries 117A-n generated therefrom. A patient summary API 174 can be utilized to enable interaction between an application 150A-n and a consolidated summary 118A-n, wherein, in an embodiment, the consolidated summary 118A-n can function as a landing page presented to the application 150A-n. Further, a Q&A API 176 can be utilized to enable interaction between an application 150A-n (e.g., per interaction with caregiver 103) and RIS 110. In an embodiment, Q&A API 176 can utilize one or more processes 127A-n to assist with processing questions 135A-n and generating answers 136A-n. In an aspect, a process 127R can be a retrieval-augmented generation (RAG) process, as implemented by RAG component 177 associated with Q&A API 176. An example RAG process being implemented in accordance with one or more embodiments is presented in FIG. 9. The Q&A API 176 enables the various components of RIS 110 to be responsive to questions 135A-n applied to any of reports 115A-n, report summaries 117A-n, and a consolidated summary 118A-n. It is to be appreciated that while only APIs 172, 174, and 176 are presented, any number of APIs can be utilized in accordance with the embodiments and functionalities presented herein regarding implementation of RIS 110 with various front-end applications 150A-n (e.g., whether those applications 150A-n are designed/operated by the RIS 110 developer or by a third party utilizing the RIS 110 functionality).

In an embodiment, application 150A-n can include a user interface (e.g., per HMI 186) whereby caregiver 103 can submit one or more questions 135A-n (a.k.a., a query transcription). Questions 135A-n can be submitted via any suitable technology, e.g., via a touchscreen 187, a keyboard (not shown), a microphone (not shown, but referenced in FIGS. 2-5), and suchlike. Question 135A-n can be submitted in the manner particular to caregiver 103, e.g., dialect, language, sentence structure, phrasing, terminology, and suchlike, enabling caregiver 103 to interact with application 150A-n and RIS 110 in as natural a manner as possible (e.g., natural language), and thus minimizing any frustration caregiver 103 may have in interacting with an automated/computer system, e.g., RIS 110. In an embodiment, the Q&A API 176 can utilize one or more processes 127A-n to parse/process the question 135A-n, e.g., LLM processing, and suchlike, to facilitate application of the content/context of the question 135A-n with the respective reports 115A-n, report summaries 117A-n, and/or consolidated summaries 118A-n. In an embodiment, the questioning user interface can be self-contained software packets (widget) such that designers/implementers of applications 150A-n can quickly and easily embed this capability into their application(s) 150A-n. Additional widgets could be provided, such as a consolidated summary widget. An additional widget could be a report-page widget showing the report summary, the report full-page, and respective metadata.

As further shown, RIS 110 can further include a prompt component 190 comprising various prompt subcomponents, for example, a report summary prompt component 192, a patient summary prompt component 194, and a summary explanation prompt component 196, configured to generate one or more prompts 197A-n. In an embodiment, as further described, prompts 197A-n can be utilized to tailor/constrain/filter information generated by summary component 125 and processes 127A-n. In another embodiment, prompts 197A-n can be utilized to configure/format/apply context to questions 135A-n to enable focused, contextual answers 136A-n to be generated. Prompts 197A-n can be previously generated and subsequently selected for implementation, e.g., a first prompt 197A is generated for implementation by a first caregiver 103A when interacting with RIS 110, while a second prompt 197B is generated for implementation by a second caregiver 103B when interacting with RIS 110. In an embodiment, the various prompts 197A-n can be self-contained software packets (e.g., comparable to a widget) such that designers/implementers of applications 150A-n can obtain respective prompts 197A-n configured for their particular requirements and incorporate the respective prompt 197A-n into their application(s) 150A-n. For example, a prompt 197D can be uniquely configured to function with an application 150D given unique requirements of the entity (e.g., caregiver 103, hospital requirements, etc.) operating application 150D.

As previously mentioned, RIS 110 can further include historical data 198A-n, wherein historical data 198A-n can comprise previously utilized patient reports 115A-n, report summaries 117A-n, consolidated summaries 118A-n, hyperlinks 113A-n, prompts 197A-n, questions 135A-n, answers 136A-n, prior interaction by one or more caregivers 103A-n with any of patient reports 115A-n, report summaries 117A-n, consolidated summaries 118A-n, hyperlinks 113A-n, prompts 197A-n, questions 135A-n, answers 136A-n, information presented on screens 187A-n and regions included therein, and suchlike. Accordingly, processes 127A-n can be trained as a function of prior interactions with particular information by a caregiver 103A-n. Hence, when a first prompt 197A was previously utilized to parse a question 135A, if further information was sought by the caregiver 103, the importance of the further information (e.g., reports similar to a patient report 115D are typically utilized by a caregiver 103A) can be established such that after repeated/separate instances of interaction, a process 127F associated with report 115D can be trained to weight/bias report 115D when presenting information (e.g., a summary) relating to report 115D on screen 187A. Further, by frequently training the respective processes 127A-n, as new information is received, e.g., research is obtained regarding a parameter/measure/criteria being determined by the medical community to be playing a greater/lesser role in diagnosing a condition, the respective processes 127A-n can be trained to reflect the greater/lesser importance of a particular criteria, etc., in diagnosing a condition, and whether information relating to the particular criteria should be included/removed during creation of the summary screen 160A-n by the summary component 120.

It is to be appreciated that the various processes 127A-n and operations presented herein are simply examples of respective AI and ML operations and techniques, and any suitable AI/ML model/technology/technique/architecture can be utilized in accordance with the various embodiments presented herein. In an aspect, processes 127A-n can operate singly or in combination to create one or more applications configured to be implemented regarding identifying a particular medical condition, e.g., a collection of processes 127A-n forming an application to identify issues relating to patient 102's cardio health. Processes 127A-n can be based on application of terms, phrases, criteria, parameters, variables, and suchlike, in patient reports 115A-n, report summaries 117A-n, consolidated summary 118A-n, historical data 198A-n, questions 135A-n, answers 136A-n, prompts 197A-n, hyperlinks 113A-n/114A-n, and suchlike. Summary component 125 can be utilized to implement processes 127-n in conjunction with RIS 110 and any components included in RIS 110. An example process 127A-n can include a vectoring technique such as bag of words (BOW) text vectors, and further, any suitable vectoring technology can be utilized, e.g., Euclidean distance, cosine similarity, etc. Other suitable AI/ML technologies/processes 127A-n that can be applied include, in a non-limiting list, any of vector representation via term frequency-inverse document frequency (tf-idf) capturing term/token frequency in any of reports 115A-n, report summaries 117A-n, consolidated summaries 118A-n, questions 135A-n, answers 136A-n, historical data 198A-n, etc. Other applicable AI/ML technologies include, in a non-limiting list, neural network embedding, layer vector representation of terms/categories (e.g., common terms having different tense), bidirectional and auto-regressive transformer (BART) model architecture, a bidirectional encoder representation from transformers (BERT) model, a diffusion model, a variational autoencoder (VAE), a generative adversarial network (GAN), a language-based generative model such as a large language model (LLM), a generative pre-trained transformer (GPT), a long short-term memory (LSTM) network/operation, a sentence state LSTM (S-LSTM), a deep learning algorithm, a sequential neural network, a sequential neural network that enables persistent information, a recurrent neural network (RNN), a convolutional neural network (CNN), a neural network, capsule network, a machine learning algorithm, a natural language processing (NLP) technique, sentiment analysis, bidirectional LSTM (BiLSTM), stacked BiLSTM, and suchlike. Accordingly, in an embodiment, implementation of the summary component 125, prompt component 190, API component 170, and suchlike, enables plain/natural language programming/annotation/correlation between any of patient reports 115A-n, report summaries 117A-n, consolidated summaries 118A-n, historical data 198A-n, questions 135A-n, answers 136A-n, prompts 197A-n, hyperlinks 113A-n/114A-n, and suchlike, to generate interactive summaries 117A-n and 118A-n, and further, questions 135A-n and answers 136A-n presented on summary screens 187A-n.

Language models, LSTMs, BARTs, etc., can be formed with a neural network that is highly complex, for example, comprising billions of weighted parameters. Training of the language models, etc., can be conducted, e.g., by summary component 125 and other components of RIS 110, etc., with datasets, whereby the datasets can be formed using any suitable technology, such as data in patient reports 115A-n, report summaries 117A-n, consolidated summary 118A-n, historical data 198A-n, questions 135A-n, answers 136A-n, prompts 197A-n, hyperlinks 113A-n/114A-n, and suchlike. Further, as previously mentioned, patient reports 115A-n, report summaries 117A-n, consolidated summaries 118A-n, historical data 198A-n, questions 135A-n, answers 136A-n, prompts 197A-n, hyperlinks 113A-n/114A-n, and suchlike, and suchlike, can comprise text, alphanumerics, numbers, single words, phrases, short statements, etc. Fine-tuning of a process 127A-n can comprise application of patient reports 115A-n, report summaries 117A-n, consolidated summary 118A-n, historical data 198A-n, questions 135A-n, answers 136A-n, prompts 197A-n, hyperlinks 113A-n/114A-n, etc., to the process 127-n, the process 127A-n is correspondingly adjusted by application of the historical data 198A-n, etc., such that, for example, weightings in the respective process 127-n are adjusted by application of the historical data 198A-n, and suchlike. As new information (e.g., report 115A-n is processed) historical data 198A-n can be updated accordingly, and further, processes 127A-n fine-tuned.

As further shown, RIS 110 can be communicatively coupled to a computer system 180. Computer system 180 can include a memory 184 that stores the respective computer executable components (e.g., summary component 125, prompt component 190 and subcomponents 192-196, API component 170 and APIs 172, 174, 176, and suchlike) and further, a processor 182 configured to execute the computer executable components stored in the memory 184. Memory 184 can further be configured to store any of patient reports 115A-n, report summaries 117A-n, consolidated summaries 118A-n, historical data 198A-n, questions 135A-n, answers 136A-n, prompts 197A-n, hyperlinks 113A-n/114A-n, AI similarity indexes S₁₋ₙ, AI vectors Vn, and suchlike. The computer system 180 can further include a human machine interface (HMI) 186 (e.g., a display, a graphical-user interface (GUI)) which can be configured to present various information including summary screens 160A-n, receive questions 135A-n, instructions to configure prompts 197A-n, mouse/cursor inputs, keyboard inputs, and suchlike. HMI 186 can include an interactive display/screen 187A-n to present the various summary screens 160A-n, reports 115A-n, report summaries 117A-n, consolidated summary 118A-n, historical data 198A-n, questions 135A-n, answers 136A-n, prompts 197A-n, hyperlinks 113A-n/114A-n, etc. Computer system 180 can further include an I/O component 188 to receive and/or transmit reports 115A-n, report summaries 117A-n, consolidated summary 118A-n, historical data 198A-n, questions 135A-n, answers 136A-n, prompts 197A-n, hyperlinks 113A-n/114A-n, and suchlike.

As further shown in FIG. 1A, system 100 can further comprise a caching component 128 (e.g., in memory 184). Caching component 128 can be configured to interface with patient summary API 174, Q&A API 176, and suchlike. Implementation of caching component 128 enables AI processing (e.g., in conjunction with processes 127A-n) for previously computed requests to be avoided. For instance, if the consolidated summary 118A-n is not cached, every time caregiver 103 accesses the patient page in an application using that API the consolidated summary would be re-computed, which can be an expensive and exhibit unnecessary delay. Similarly, if different caregivers 103 pose the same question 135A-n to the Q&A API 176, caching component can provide the answer without delay or extra cost.

Various communications 198A-n can be utilized across the system 100/100A, between RIS 110 (and included components), applications 150A-n, EMR system 105, computer system 180, etc. Communications 198A-n can include notifications, instructions, status updates, selections, data, information (e.g., questions 135A-n, answers 136A-n, etc.), and the like.

FIG. 1B presents a schematic 100A of report summaries being generated from various initial reports, and a consolidated report being generated from the report summaries, according to at least one embodiment. Generation of the consolidated summary 118A is presented in a series of steps.

At FIG. 1B-1, as previously mentioned, a collection of patient reports 115A-n are available for patient 102.

At FIG. 1B-2, as previously mentioned, a computer-implemented process 127A-n (e.g., an LLM) can be utilized to generate report summaries 117A-n, and further implemented to generate consolidated reports 118A-n. Further, prompts 197A-n can be applied to the process, e.g., process 127A, to enable generation of report summaries 117A-n in accordance with an L1 or L2 level format. Accordingly, one or more report summaries 117A-n can be generated from the patient reports 115A-n, for example, a report summary 117A-L1 is generated from patient report 115A, report summary 117B-L1 is generated from patient report 115B, report summary 117n-L1 is generated from patient report 115n, with each of report summary 117A-L1, report summary 117B-L1, and report summary 117n-L1 being limited to 10 words or less, in accordance with the configuration of L1, as previously mentioned. Further, the report summaries 117A-n having an L1 format can be utilized to provide short statement summaries of a patient report 115A-n (e.g., per FIG. 5, links 113A-n having an L1 format).

At FIG. 1B-3, computer-implemented process 127A-n can be further utilized to generate report summaries 117A-n having an L2 format (e.g., summarized with between 10 and 150 words). Accordingly, one or more report summaries 117A-n can be generated from the patient reports 115A-n, whereby, a report summary 117A-L2 is generated from patient report 115A, report summary 117B-L2 is generated from patient report 115B, report summary 117n-L2 is generated from patient report 115n, with each of report summary 117A-L2, report summary 117B-L2, and report summary 117n-L2 comprising between 10 and 150 words, in accordance with the configuration of L2, as previously mentioned. Further, the report summaries 117A-n having an L2 format can be utilized to provide longer statement summaries of a patient report 115A-n (e.g., per FIG. 5, link 113L having an L2 format).

At FIG. 1B-4, a consolidated summary 118A can be generated from the report summaries 117A-n. For example, a consolidated summary 118A can be generated from the report summaries 117A-n created with the L2 format. Accordingly, the prompt 197C assigned to creating consolidated summary 118A can be configured to also constrain the consolidated summary 118A to a L2 format. In a further example embodiment, prompt 197L can be configured to generate the consolidated summary 118A based on a particular parameter, e.g., a date of a last visit by patient 102 with caregiver 103, and the prompt 197L is configured to generate a consolidated report 118L summarizing any new patient reports 115A-n that have been generated since the last visit with caregiver 103 (e.g., information obtained, test results, etc., performed since the last visit) to enable the caregiver 103 to identify the outcome of a recommended treatment, examination, etc., since patient 102's last visit.

FIG. 1C presents a schematic 100B of report summaries being generated from various initial reports, and a consolidated summary being generated from the report summaries, according to at least one embodiment. Generation and implementation of the consolidated summary 118A is presented in a series of steps.

At FIG. 1C-1, as previously mentioned, a variety of patient reports 115A-n are available for a patient 102, whereby, the various patient reports 115A-n can be separated out into respective common subject matter, themes, data, etc., such that patient reports 115A-C have a first common theme (e.g., oncology) and reports 115D-G have a second common theme (e.g., radiology). In an embodiment, the summary component 125 can be configured to utilize any suitable computer-based model, e.g., a process 127A-n comprising, for example, a large language model (LLM) to identify respective content in patient reports 115A-n to enable the various patient reports 115A-n to be separated into common subject matter. Furthering the example, the content (e.g., chunks, statements, etc.) of respective patient reports 115A-n can be compared with a particular theme/subject matter, e.g., oncology, radiology, and the like, wherein similarity of the content can be assessed based on vectored representations and similarity, substantial similarity, threshold similarity, and the like, between the respective content and the particular theme/subject matter (e.g., as further described with regard to FIG. 9).

At FIGS. 1C-2A and 1C-2B respectively, the common groups of reports (e.g., first group of patient reports 115A-C and second group of reports 115D-G) can have respective summary reports generated therefrom, such that the first group of patient reports 115A-C are summarized in first report summary 117A and the second group of patient reports 115D-G are summarized in second report summary 117B. In an embodiment, the summary component 125 can be configured to utilize any suitable computer-based model, e.g., a process 127A-n comprising, for example, a LLM configured to identify respective content in the respective patient reports 115A-n to enable generation of the respective summary reports 117A-n. Furthering the example, the content (e.g., chunks, statements, etc.) of respective patient reports 115A-n can be compared with a particular theme/subject matter, e.g., oncology, radiology, and the like, wherein similarity of the content can be assessed based on vectored representations and similarity, substantial similarity, threshold similarity, and the like, between the respective content and the particular theme/subject matter (e.g., as further described with regard to FIG. 9). Further, a particular summary 117A-n is not limited to a particular theme, but can be generated based on a series of terms/parameters, e.g., a medical condition, age-related illness that may pertain to the patient 102, and suchlike. The particular terms/parameters can be adjusted in accordance with knowledge of a medical condition, knowledge about patient 102, and the like.

At FIG. 1C-3, the content of the first report summary 117A the content of the second report summary 117B are combined to form the consolidated summary 118A. In an embodiment, the summary component 125 can be configured to utilize any suitable computer-based model, e.g., a process 127A-n comprising, for example, a LLM configured to identify respective content in the respective summaries 117A-n to enable generation of the consolidated summary 118A-n. Furthering the example, the content (e.g., chunks, statements, etc.) of respective summary reports 117A-n can be compared with a particular theme/subject matter, e.g., oncology, radiology, and the like, wherein similarity of the content can be assessed based on vectored representations and similarity, substantial similarity, threshold similarity, and the like, between the respective content and the particular theme/subject matter (e.g., as further described with regard to FIG. 9). Further, a consolidated summary 118A-n is not limited to a particular theme, but can be generated based on a series of terms/parameters, e.g., a medical condition, age-related illness that may pertain to the patient 102, and suchlike. The particular terms/parameters can be adjusted in accordance with knowledge of a medical condition, knowledge about patient 102, and the like.

Through steps 1-3, any of the patient reports 115A-n, report summaries 117A-n, and/or consolidated summary 118A, can be presented on HMI 186, for viewing, interaction, and selection of links 113A-n/114A-n.

At FIG. 1C-4A, selection of a hyperlink 113A at a consolidated report 118A can cause a report summary 117A or 117B associated with the selected link to be presented.

At FIG. 1C-4B, as previously mentioned, a hyperlink 114A-n can also link directly to the patient report 115A-n, hence, selection of a hyperlink 114A presented at a consolidated report 118A can cause the associated patient report 115A-n to be presented.

At FIG. 1C-5, a hyperlink 114A-n presented at a report summary 117A-n can cause the associated patient report 115A-n to be presented.

Hence, per steps 1-5, patient reports 115A-n can be summarized as report summaries 117A-n, further as a consolidated summary 118A-n, and per selection of links 113A-n/114A-n, the patient reports 115A-n/report summaries 117A-n can also be navigated to and presented. As mentioned, as complexity of available patient reports 115A-n increases, presenting summarized information to caregiver 103 may utilize two or more consolidated summaries 118A-n being concurrently presented, with each consolidated summary 118A-n being of a specific technology/field, e.g., oncology, radiology, etc. It is to be appreciated that the processes 127A-n (e.g., an LLM, vectorization, retrieval-augmented generation (RAG) processing, and the like), can be applied at any of the operations/embodiments presented herein, e.g., regarding processing of the patient reports 115A-n, generation of the summary report 117A-n, generation of the consolidated summary 118A-n, interaction with hyperlinks 113A-n/114A-n, application of queries 135A-n, generation of answers 136A-n, implementation/updating of prompts 197A-n, and the like.

FIG. 1D presents a schematic 100C of reports being generated over the course of a series of visits, and a summary report being generated to summarize activity, patient update, etc., for the respective dates of visits and intervening actions, according to at least one embodiment.

As shown, patient 102 is under the medical guidance of an oncologist 103, whereby at FIG. 1D, step 1, on Jan. 1, 2024, patient 102 first visits the oncologist 103. During the first visit, oncologist 103 instructs patient 102 to have a first exam (e.g., blood work, etc.), a first biopsy, and a first MRI examination.

At FIG. 1D, step 2, a second patient report 115B can be generated and includes the results of the first exam, the first biopsy, and the first MRI examination, etc.

At FIG. 1D, step 3, on Mar. 1, 2024, patient 102 visits oncologist 103 for a second time. During the second visit, the oncologist 103 can utilize RIS 110 to generate a first summary report 117A (per FIG. 1D, step 3A), whereby the summary report 117A summarizes any updates, activities, etc., regarding patient 102 between Mar. 1, 2024 and Jan. 1, 2024. Accordingly, first summary report 117A can be a summarization of differences/updates in the second patient report 115B and the first patient report 115A. During the second visit, oncologist 103 instructs patient 102 to have a second exam (e.g., blood work, etc.), a second biopsy, and a second MRI examination.

At FIG. 1D, step 4, a third patient report 115C can be generated and includes the results of the second exam, the second biopsy, and the second MRI examination, etc.

At FIG. 1D, step 5, on Jun. 1, 2024, patient 102 visits oncologist 103 for a third time. During the third visit, the oncologist 103 can utilize RIS 110 to generate a second summary report 117B (per FIG. 1D, step 5A), whereby the second summary report 117B summarizes any updates, activities, etc., regarding patient 102 between Jun. 1, 2024 and Mar. 1, 2024. Accordingly, second summary report 117B can be a summarization of differences/updates in the third patient report 115C and the second patient report 115B. Further, during the third visit, the oncologist 103 can utilize RIS 110 to generate a third summary report 117C (per FIG. 1D, step 5B), whereby the third summary report 117C summarizes any updates, activities, etc., regarding patient 102 between Jun. 1, 2024 and Jan. 1, 2024. Accordingly, third summary report 117C can be a summarization of differences/updates in the third patient report 115C and the first patient report 115A.

Per the foregoing, a summary report 117A-n can be a summarization of patient activity, information, etc., between two or more dates, e.g., Jun. 1, 2024 and Mar. 1, 2024; Mar. 1, 2024 and Jan. 1, 2024; Jun. 1, 2024 and Jan. 1, 2024, and the like.

### Prompts

As previously mentioned, a prompt component 190 can be configured to generate various prompts 197A-n which are used as input to an AI model (e.g., process 127A is an LLM) to instruct the AI model to return the required information (e.g., a report summary 117A-n, a patient summary (e.g., consolidated summary 118A-n) or the answer to a question). Prompts 197A-n can be further utilized to control/structure how information, images, etc., are compiled in the report summaries 117A-n and consolidated summaries 118A-n and further, prompts 197A-n can be utilized to generate focused interaction with a Q&A API 176. In an aspect, prompts 197A-n can be further utilized to constrain information such that only information that complies with a prompt 197A-n is presented/utilized, which can cause caregiver 103 to have greater confidence in the information selected/presented as part of summaries 117A-n/118A-n, and further, create answers 136A-n that are accurate, unambiguous, and have supportable content.

In an embodiment, prompts 197A-n can be generated by a report summary prompt component 192 configured to implement prompts 197A-n directed towards analysis of report summaries 117A-n from the respective reports 115A-n. In another embodiment, a patient summary prompt component 194 can be configured to generate prompts 197A-n for implementation with the consolidated summaries 118A-n. For example, rather than the summary component 125 simply processing questions 135A-n from caregiver 103, the summary component 125 can utilize prompts 197A-n (and processes 127A-n) to supplement/construct one or more questions 135A-n formulated in accordance with prompts 197A-n, where, in effect, the prompts 197A-n can function as a query template, with the prompts 197A-n providing context, structure, etc., regarding how process 127A-n (e.g., an LLM) is to identify information (e.g., in summaries 118A-n/117A-n, reports 115A-n, historical data 198A-n, and suchlike) pertinent to a question 135A-n for creation and presentation as an answer 136A-n. In another example, prompts 197A-n can be utilized to apply context to a questions 135A-n, e.g., only provide answers 136A-n that have explicit support in the patient reports 115A-n, if no answer found then do not provide unsourced answer, and suchlike. In another embodiment, a summary explanation prompt component 196 can be configured to generate prompts 197A-n for generation and linking of hyperlinks 113A-n/114A-n.

### Example Screens

To provide further understanding of the various embodiments presented herein, FIGS. 2-5, images 200-500 present screen captures of a series of example screens, in accordance with one or more embodiments.

FIG. 2, screen 200, illustrates an example screen that may be presented by a radiology application, in accordance with an embodiment. Screen 200 presents an example summary screen 160A which can be presented (e.g., by presentation component 126) on display 187 and, as shown, can comprise a first screen portion 210 and a second screen portion 220, wherein screen portion 210 can be utilized to present respective images 230A-n regarding patient 102's medical condition, while screen portion 220 can be utilized to present respective one or more summaries 117A-n/118A-n, and further, when a hyperlink 113A-n is selected, to present report 115A-n (e.g., as a pop-up screen) as well as navigating between the summaries 117A-n/118A-n. In an example implementation, screen 200 can be a screen developed for radiology review, wherein the API component 170 can be configured to be an interface for screen 160A/RIS 110 and a radiology application 150A. In an embodiment, presentation component 126 and/or patient summary API 174 can be configured to present the summary screen 200 including images, summaries 117A-n/118A-n, hyperlinked 113/114 information, etc. Screen 200 can be configured to present specific information, e.g., radiology-only information, oncology-only information, etc., or a combination of multiple disciplines as required to diagnose/treat patient 102, e.g., screen 200 provides a combination of radiology and oncology information.

In an embodiment, screen 200 can be an initial screen/landing page, whereby medical information pertaining to patient 102 can be presented utilizing a consolidated summary 118A-n, from which caregiver 103 can navigate through the information as required to review/analyze patient 102's medical condition. The consolidated summary 118A-n can be further presented with one or more hyperlinks 113A-n, connecting, as previously mentioned, a consolidated summary 118A-n with the report summaries 117A-n from which consolidated summary 118A-n was generated. Accordingly, by selecting a hyperlink 113A-n, caregiver 103 can temporarily review a report summary 117A-n pertaining to a particular finding, comment, measurement, etc., presented in the consolidated summary 118A-n, wherein the report summary can be presented in a pop-up window presented over screen 200. In another embodiment, by selecting hyperlink 113A-n, the respective report summary 117A-n can be presented as a window on screen 160A-n.

Further, report summary 117A-n can be presented with one or more hyperlinks 114A-n connecting, as previously mentioned, a report summary 117A-n with the report 115A-n which is summarized by report summary 117A-n. Accordingly, caregiver 103 can review patient information presented on screen 160A-n, and navigate up/down between any of the consolidated summary 118A-n, report summaries 117A-n, and/or reports 115A-n. In an aspect, the respective hyperlinks 113A-n and 114A-n can relate to a particular item of information/fact in an underlying report 115A-n or report summary 117A-n, e.g., from where the item of information/fact originates. For example, if report summary 117A-n states that patient 102 underwent a surgery, the hyperlink 113A or 114A stating the surgery can be hyperlinked to the surgery report 115S, enabling caregiver 103 to a) verify that the surgery information is correct, and b) follow-up on surgery information if required.

As further shown on FIG. 2, screen 200 can include a microphone icon 260, whereupon when caregiver 103 selects icon 260, a microphone/voice interface can be activated at HMI 186, enabling caregiver 103 to submit one or more questions 135A-n to the RIS 110, via the application 150A-n and any suitable interface to facilitate the interaction, e.g., a chat interface, a chatbot, and suchlike. Hence, caregiver 103 can interact with screen 200 via voice commands, queries, etc., and further via keyboard.

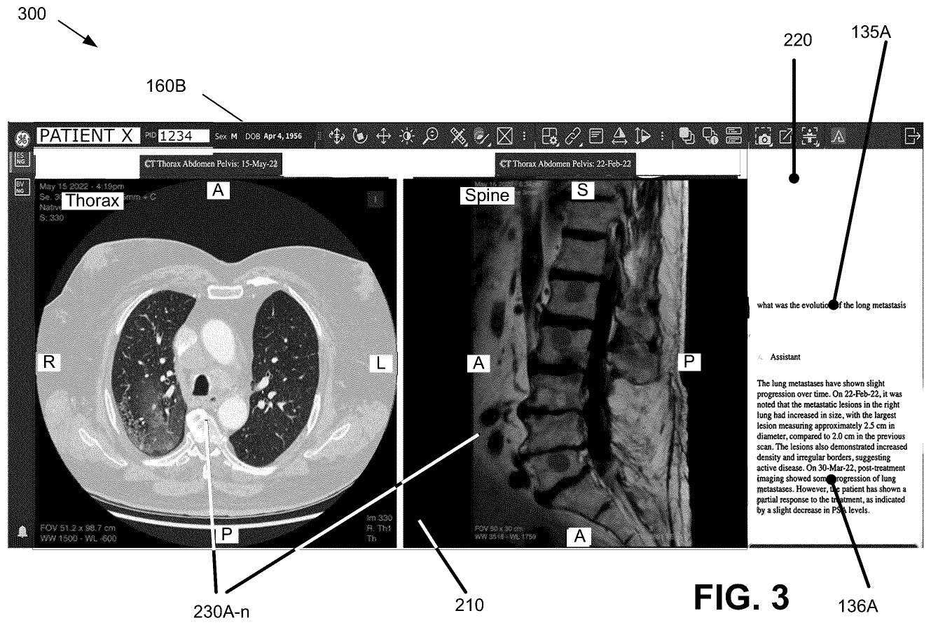

FIG. 3, screen 300, illustrates an example screen that may be presented by a radiology application, in accordance with an embodiment. Screen 300 illustrates the text of consolidated summary 118A-n (per FIG. 2) being replaced by a question 135A and a response 136A provided to the question 135A. In another embodiment, the region (e.g., screen portion 220) displaying the content of the consolidated summary 118A-n can be kept visible, and the question 135A and answer 136A can scroll either above or below the consolidated summary 118A-n. As further described, in an embodiment, the question 135A can be processed, e.g., by summary component 125 in conjunction with processes 127A-n/Q&A API 176, wherein the response 136A can be a result of a LLM process or suchlike identifying terms/context in the question 135A and applying those terms/context directly to any of reports 115A-n, report summaries 117A-n, consolidated summaries 118A-n, with the answer 136A being identified (e.g., by summary component 125) as being relevant to question 135A. In another embodiment, question 135A can be received (e.g., by an interface at I/O 188), and further have a prompt 197A-n applied to question 135A, wherein the prompt 197A-n can limit scope of the question 135A, answering the question 135A with the most recent data available first. The prompted 197A-n version of then originally received question 135A.

Hence, in an embodiment, the processing ability of summary component 125 (in conjunction with processes 127A-n) is improved over a conventional system as a function of the question 135A being applied to one or both of summaries 117A-n and/or 118A-n, whereby the summaries 117A-n/118A-n are a distillation of the reports 115A-n, and accordingly, summaries 117A-n/118A-n are potentially smaller files than the reports 115A-n. And further, with prompts 197A-n functioning to structurally limit the number of terms/scope of a question 135A, processing time of the question 135A should be significantly faster than a conventional, prompt-less, non-summary based system.

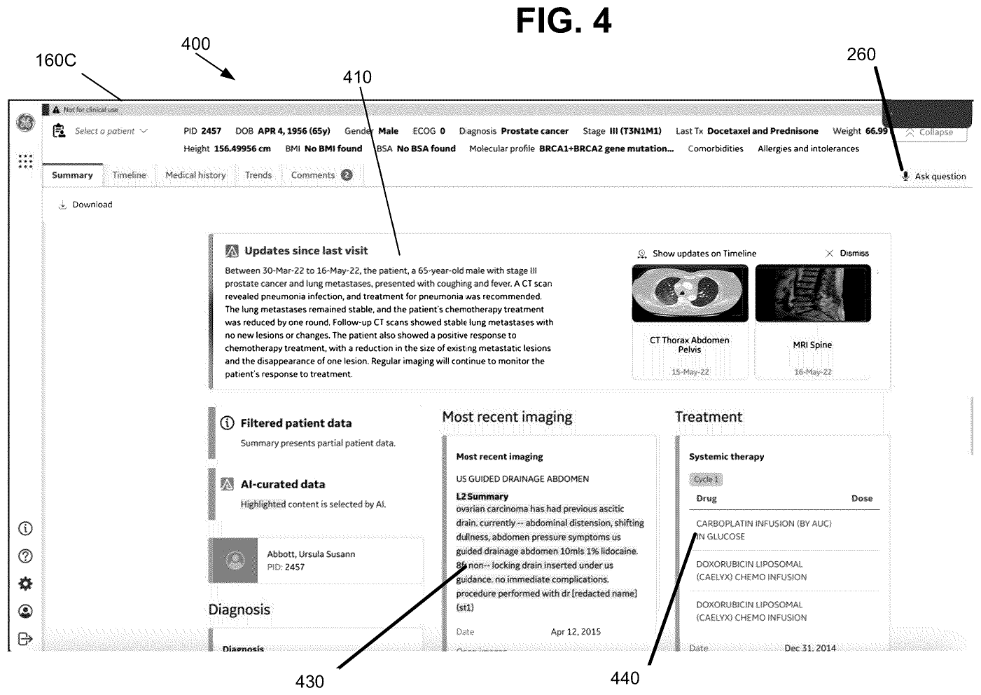

FIG. 4, screen 400, illustrates an example screen that may be presented by an oncology application, in accordance with an embodiment. As previously mentioned, screen 400 (screen 160C) can be generated by an application 150C (e.g., a local party application or third party application), by RIS 110 for implementation with an oncology application 150C, and suchlike. Screen 400 has similarities to screen 200 with regard to presenting summaries 117A-n/118A-n, e.g., summary provision and Q&As. However, while screen 200 was providing summaries 117A-n/118A-n based on all the available information for patient 102, with screen 400, a summary 118A-n can be based on what happened during/since last visit, per summary's 410 and 430 (e.g., derived from either of 117A-n/118A-n), and information 440 regarding systemic therapy. As further shown on FIG. 4, screen 400 can include a microphone icon 260, as previously described.

FIG. 5, screen 500, illustrates an example screen that may be presented by an oncology application, in accordance with an embodiment. As previously mentioned, screen 500 (screen 160D) can be generated by an application 150C (e.g., a local or third party application), by RIS 110 for implementation with an oncology application 150C, and suchlike. As shown on screen 500, a timeline 510 can be presented with dots/icons 520A-n indicating when particular reports 115A-n were generated (e.g., patient visit, medical procedure, etc.), whereby, selection of a dot 520A-n can cause a particular report 115A-n to be presented pertaining to the dot 520A-n In an embodiment, with the dot selected, the report summary 117A-n for that particular report 115A-n can be presented in summary region 530, as well as direct selection 540 of the report 115A-n.

As further shown, on FIG. 5, a series of links 113A-n/114A-n are presented having an L1 format, whereby the links have ten or fewer words. In an embodiment, as a cursor is placed over any of dots 520A-n, the corresponding link 113A-n/114A-n can be highlighted to enable determination and selection of a report summary 117A-n/patient report 115A-n corresponding to the particular dot 520A-n.

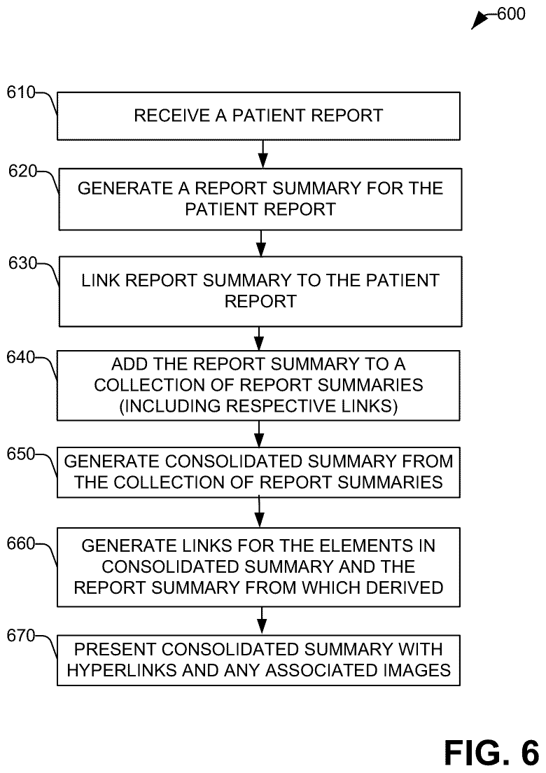

FIG. 6, via flowchart 600, presents an example computer-implemented method for generating a summary page from a patient report, in accordance with one or more embodiments.

At 610, a patient report 115A-n can be received at a reporting system (e.g., RIS 100).

At 620, a report summary 117A can be automatically generated (e.g., by summary component 125 in conjunction with processes 127A-n) from the patient report 115A-n, wherein report summary 117A can be generated from content in patient report 115A-n.

At 630, report summary 117A can be linked (e.g., by report summary component 125 generating a hyperlink 114A) to the patient report 115A-n from which report summary 117A was generated.

At 640, the report summary 117A can be added (e.g., by summary component 125) to a collection of report summaries 117B-n. In an example implementation, the report summaries 117A-n can all pertain to the same patient (e.g., patient 102). Hyperlinks 114A-n can be provided (e.g., by summary component 125) to enable navigation between respective patient reports 115A-n and associated report summary 117A-n.

At 650, a consolidated summary 118A-n can be generated (e.g., by summary component 125) from the collection of report summaries 117A-n.

At 660, hyperlinks 113A-n can be generated (e.g., by summary component 125) linking respective content in the consolidated summary 118A-n with the source report summary 117A-n that generated the respective information.

At 670, the consolidated summary 118A can be presented (e.g., by presentation component 126, on HMI 186, display 187, summary screens 160A-n).

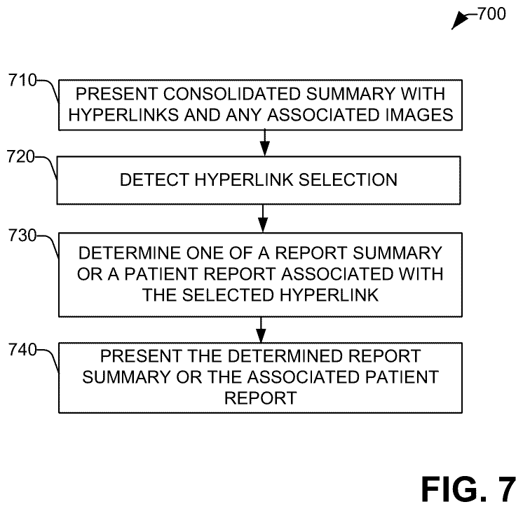

FIG. 7, via flowchart 700, illustrates an example computer-implemented method for accessing further data associated with a summary page, in accordance with one or more embodiments.

At 710, a consolidated summary 118A can be presented (e.g., on HMI 186, display 187, summary screens 160A-n). The consolidated summary 118A can include images 230A-n and text for consolidated summary 118A. One or more hyperlinks 114A-n/113A-n can be present in the consolidated summary 118A (e.g., as populated by summary component 125).

At 720, selection of a hyperlink 114A-n can be detected (e.g., by presentation component 126), e.g., as a function of caregiver 103 interacting with the consolidated summary 118A. As previously mentioned, a hyperlink in the text of the consolidated summary 118A presented on a screen can link back to an underlying report summary 117A-n from which the hyperlinked text was derived.

At 730, the source of the information in the selected hyperlink 113A can be determined (e.g., by the summary component 125).

At 740, the report summary 117A-n associated with the selected hyperlink 113A can be presented on the screen (e.g., by presentation component 126). In another embodiment, the hyperlink 114A-n can be between information presented on a report summary 117A-n, whereby selection of the hyperlink 114A will cause a patient report 115A to be presented (e.g., by presentation component 126).

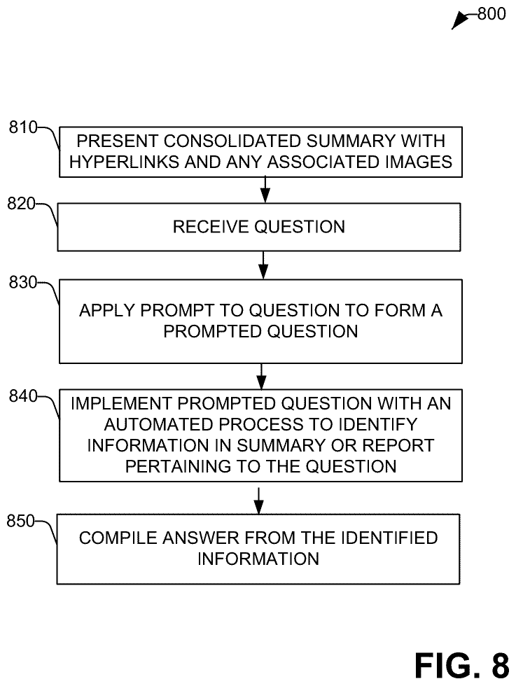

FIG. 8, via flowchart 800, illustrates a computer-implemented method for applying a prompt to a question to constrain scope of question and/or supplied answer, in accordance with an embodiment.

At 810, a consolidated summary 118A can be presented (e.g., by presentation component 126) on a summary screen 160A, in conjunction with associated images 230A-n and hyperlinks 113A-n.

At 820, a question (e.g., a question 135A-n) can be received (e.g., at a chatbot implemented on an HMI 186 on which the summary screen 160A is being presented).

At 830, a prompt 197A-n can be applied to the question 135A-n (e.g., by prompt component 190), wherein the prompt 197A-n can be configured to control how an answer 136A-n to the question 135A-n is to be derived/presented, e.g., present most recent pertinent data, etc.

At 840, depending upon the level of interaction, from a report or a summary (e.g., consolidated summary 118A, report summary 117A, or report 115A) information can be identified (e.g., by any of prompt component 190, summary component 125, processes 127A-n) that pertains to the prompt-modified question 135A-n.

At 840, an answer 136A-n to the question 135A-n can be generated (e.g., by any of prompt component 190, summary component 125, processes 127A-n) and presented (e.g., on screen 160A-n), wherein the answer 136A-n can be compiled from information meeting the criteria of the question 135A-n, as modified by prompt 197A-n.

### Rag Process

FIG. 9 presents an example schematic of a RAG process flow 900 as can be implemented as part of a Q&A process, in accordance with an embodiment. FIG. 9 presents a high-level overview of Q&A interaction with various documents available in a RIS 110. At 910, as shown, various documents can be received at RIS 110, e.g., a first collection of reports 115A-n, report summaries 117A-n, consolidated summaries 118A-n, pertaining to a first patient 102A, a second collection of reports 115A-n, etc., pertaining to a second patient 102B, etc. Various processes 127A-n can be applied to the reports 115A-n, and summaries 117A-n and 118A-n to enable indexing of the respective reports and summaries 115A-n, 117A-n, and 118A-n, wherein indexing can include generation of metadata associated with the respective document, separation/splitting of the document(s) into respective paragraphs/chunks pertaining to particular subject matter (e.g., regarding patient 102A-n in patient reports 115A-n, report summaries 117A-n, consolidated summaries 118A-n, and the like), with the respective paragraphs/chunks represented in an embedding space.

At 920, the respective paragraphs/chunks can be vectorized and stored in a vector store (e.g., in database 112).

A 930, a question 135A-n can be received (e.g., at RIS 110 via Q&A API 176) from caregiver 103 regarding a patient 102A-n.

At 940, question 135A-n can be query processed (e.g., via Q&A API 176) including query embedding.

At 950, question 135A-n can be processed/filtered (e.g., at Q&A API 176, RAG component 177) to determine any of content, context, and suchlike for question 135A-n, whereby the question 135A-n can be vectorized and applied to the vector store (e.g., database 112). Similarity algorithms (e.g., by summary component 125, processes 127A-n) can be applied to the vectorized questions 135A-n and the vectorized paragraphs/chunks, with content having a high degree of similarity identified and produced, aka identified content. Hence, the wealth of information in any of reports and summaries 115A-n, 117A-n, and 118A-n is being reduced to information that pertains to the content/context of question 135A-n, which if performed correctly, can reduce the processing burden on RIS 110.

At 960, the identified content can be concatenated with question 135A-n and any associated metadata to generate a prompt 197A-n.

At 970, the prompt 197A-n can be applied to one or more of the processes 127A-n, along with any training data to determine that the process 127A-n is performing correctly.

At 980, results generated by the processes 127A-n can be verified to confirm that the process 127A-n is performing correctly (e.g., generating an anticipated result) or not.

As used herein, the terms “infer”, “inference”, “determine”, and suchlike, refer generally to the process of reasoning about or inferring states of the system, environment, and/or user from a set of observations as captured via events and/or data. Inference can be employed to identify a specific context or action, or can generate a probability distribution over states, for example. The inference can be probabilistic-that is, the computation of a probability distribution over states of interest based on a consideration of data and events. Inference can also refer to techniques employed for composing higher-level events from a set of events and/or data. Such inference results in the construction of new events or actions from a set of observed events and/or stored event data, whether or not the events are correlated in close temporal proximity, and whether the events and data come from one or several event and data sources.

Per the various embodiments presented herein, various components included in RIS 110, presentation component 126, prompt component 190, API component 170, and suchlike, can include AI/ML and reasoning techniques and technologies (e.g., processes 127A-n) that employ probabilistic and/or statistical-based analysis to prognose or infer an action that a user desires to be automatically performed. The various embodiments presented herein can utilize various machine learning-based schemes for carrying out various aspects thereof. For example, a process 127A-n (e.g., by summary component 125, prompt component, patient summary API 174) for automatically determining content in report summaries 117A-n to include in consolidated summaries 118A-n; a process 127A-n (e.g., by summary component 125, prompt component, patient summary API 174) for automatically determining content in patient reports 115A-n to include in report summaries 117A-n; a process 127A-n (e.g., by summary component 125, prompt component 190, Q&A API 176, RAG component 177, etc.) for automatically determining content/an answer 136A-n to present to a question 135A-n, and suchlike, as previously mentioned herein, can be facilitated via an automatic classifier system and process.

A classifier is a function that maps an input attribute vector, x=(x1, x2, x3, x4, xn), to a class label class(x). The classifier can also output a confidence that the input belongs to a class, that is, f(x)=confidence(class(x)). Such classification can employ a probabilistic and/or statistical-based analysis (e.g., factoring into the analysis utilities and costs) to prognose or infer an action that a user desires to be automatically performed (e.g., identifying respective features presented in consolidated summaries 118A-n, report summaries 117An, patient reports 115A-n, questions 135A-n, answers 136A-n, and operations related thereto).

A support vector machine (SVM) is an example of a classifier that can be employed. The SVM operates by finding a hypersurface in the space of possible inputs that splits the triggering input events from the non-triggering events in an optimal way. Intuitively, this makes the classification correct for testing data that is near, but not identical to training data. Other directed and undirected model classification approaches include, e.g., naïve Bayes, Bayesian networks, decision trees, neural networks, fuzzy logic models, and probabilistic classification models providing different patterns of independence can be employed. Classification as used herein is inclusive of statistical regression that is utilized to develop models of priority.

As will be readily appreciated from the subject specification, the various embodiments can employ classifiers that are explicitly trained (e.g., via a generic training data) as well as implicitly trained (e.g., via observing user behavior, receiving extrinsic information). For example, SVM's are configured via a learning or training phase within a classifier constructor and feature selection module. Thus, the classifier(s) can be used to automatically learn and perform a number of functions, including but not limited to determining according to predetermined criteria, content in report summaries 117A-n to create consolidated summaries 118A-n, content/context of questions 135A-n, content in patient reports 115A-n, report summaries 117A-n, consolidated summaries 118A-n, content/instructions in prompts 197A-n, links 113/114, and generate meaningful content to present in answers 136A-n to questions 135A-n for example.

As described supra, inferences can be made, and automated operations performed, based on numerous pieces of information. For example, whether sufficient context is available to infer, with a high degree of confidence, a correlation between content in a consolidated summary 118A-n pertains to a question 135A-n, effective linking 113/114 of terms in a summary with the source content, determining content in any of patient reports 115A-n, report summaries 117A-n, consolidated summaries 118A-n, and suchlike, for presentment as answer 136A-n, and suchlike, to enable a caregiver 103 readily interpret patient information presented on summary screens 160A-n.

In review, the various embodiments presented herein enable:
a) utilization of AI/ML-based summarization and question-answering capabilities as a healthcare system and a corresponding workflow that can be used in different medical applications.
b) the usage of hyperlinks as mitigation for failure(s) in accuracy of AI/ML-since the hyperlink quickly enables the user to fact check the presented summary/patient info.
c) user experience/user interface elements that are embedded in the clinical (e.g., radiological, oncological) workflow, and can be reusable, such as a summary box and microphone-enabled querying widget.

The various embodiments utilize automated and dynamic information summarization and a Q&A system combining information from the patient record, summarizes the important events from the patient's history, and enables the caregiver 103 to ask questions about the information contained in the patient's records. In an example, a LLM can be utilized via a series of dedicated prompts.

The summaries 118A-n/117A-n provided are hyperlinked to the reports 115A-n. More specifically, each fact in the summary 118A-n/117A-n s hyperlinked to the specific report(s) 115A-n from which that fact originates. E.g., if the summary 118A states that patient 102 underwent a surgery, that part of the summary 118A is hyperlinked 113A to the surgery report 115A, enabling the caregiver 103 to a) verify that information is correct, and b) follow-up on that information.

The Q&A module can be done by speech, or by typing the question into a box.

The various embodiments presented herein enable numerous customer/patient benefits as a function of caregivers 103 being able to readily access patient information, via two phases/processes:
Patient summarization (e.g., per patient reports 115A-n, report summaries 117A-n, consolidated summaries 118A-n, and the like)—enables the caregiver 103 to understand the patient 102 status in just a few seconds.
Question-answering (e.g., per questions 135A-n, answers 136A-n, prompts 197A-n)—enables the caregiver 103 to dip-dive quickly and conveniently into what caregiver 103 deems relevant in order to complete the task of assisting patient 102.

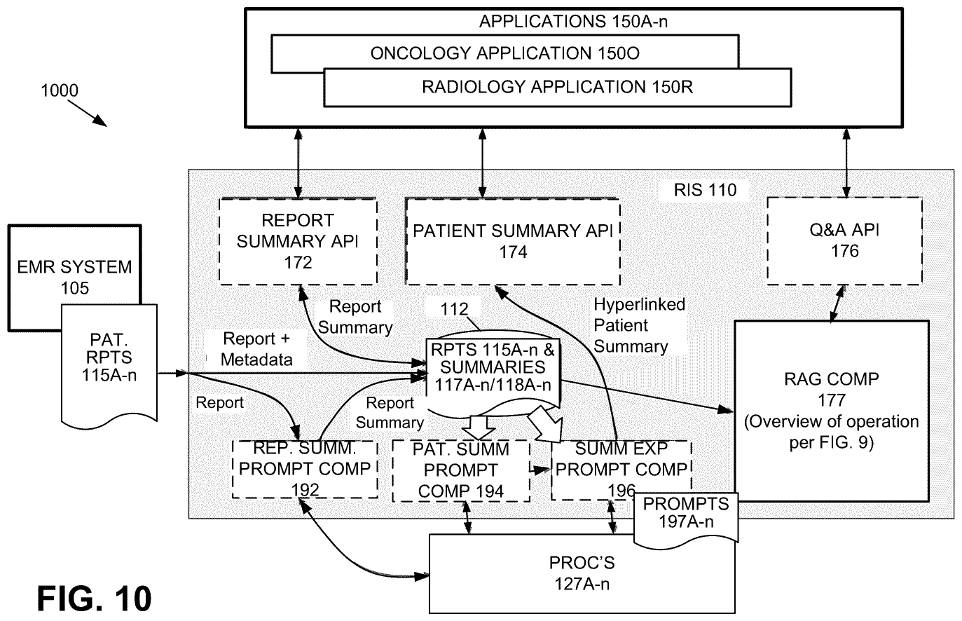

FIG. 10, schematic 1000, illustrates respective operational flow during implementation of RIS 110, in accordance with an embodiment. FIG. 10 presents respective process flow pertaining to reports 115A-n, summaries 117A-n/118A-n, questions 135A-n, answers 136A-n, prompts 197A-n, links 113A-n/114A-n, and suchlike. Review of FIG. 10 can be conducted with the respective components, etc., as described in FIGS. 1A-1D.

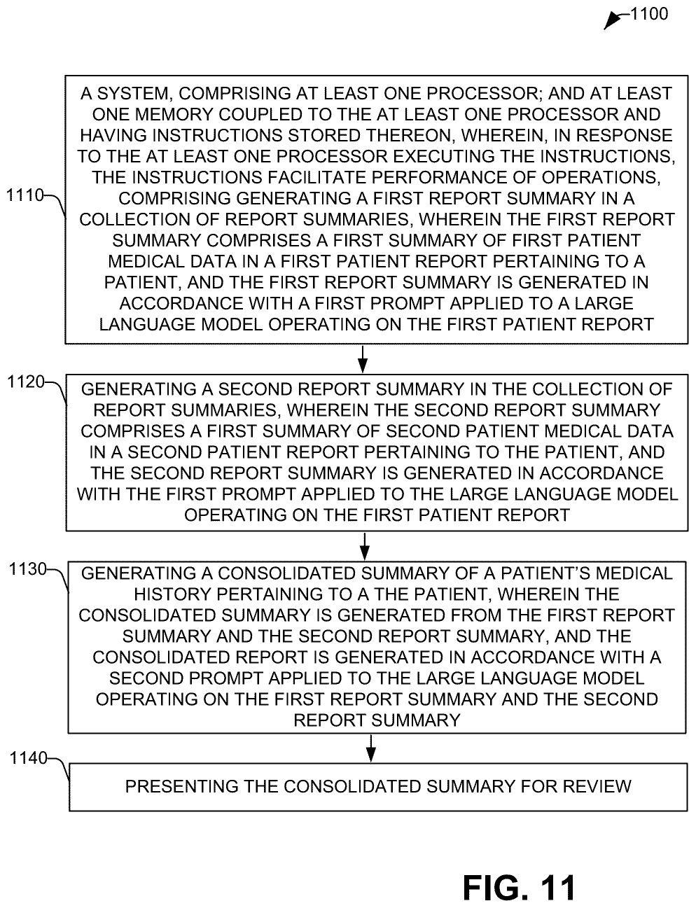

FIG. 11, via flowchart 1100, presents an example computer-implemented method for automatically and dynamically generating one or more consolidated summaries comprising and linked to summary reports and further to patient reports, in accordance with an embodiment.

At 1110, method 1100 can be performed by a system (e.g., RIS 110), comprising at least one processor (e.g., processor 182A-n) and at least one memory (e.g., memory 184A-n) coupled to the at least one processor and having instructions stored thereon, wherein, in response to the at least one processor executing the instructions, the instructions facilitate performance of operations, comprising generating a first report summary (e.g., report summary 117A) in a collection of report summaries, wherein the first report summary comprises a first summary of first patient medical data in a first patient report (e.g., patient report 115A) pertaining to a patient (e.g., patient 102), and the first report summary is generated in accordance with a first prompt (e.g., prompt 197A) applied to a large language model (e.g., process 127A) operating on the first patient report.

At 1120, the operations of method 1100 can further comprise generating a second report summary (e.g., report summary 117B) in the collection of report summaries, wherein the second report summary comprises a first summary of second patient medical data in a second patient report (e.g., patient report 115B) pertaining to the patient, and the second report summary is generated in accordance with the first prompt applied to the large language model operating on the first patient report.

At 1130, the operations of method 1100 can further comprise generating a consolidated summary (e.g., consolidated summary 118A-n) of a patient's medical history pertaining to the patient, wherein the consolidated summary is generated from the first report summary and the second report summary, and the consolidated report is generated in accordance with a second prompt (e.g., prompt 197B) applied to the large language model operating on the first report summary and the second report summary.

At 1140, the operations of method 1100 can further comprise presenting the consolidated summary for review.

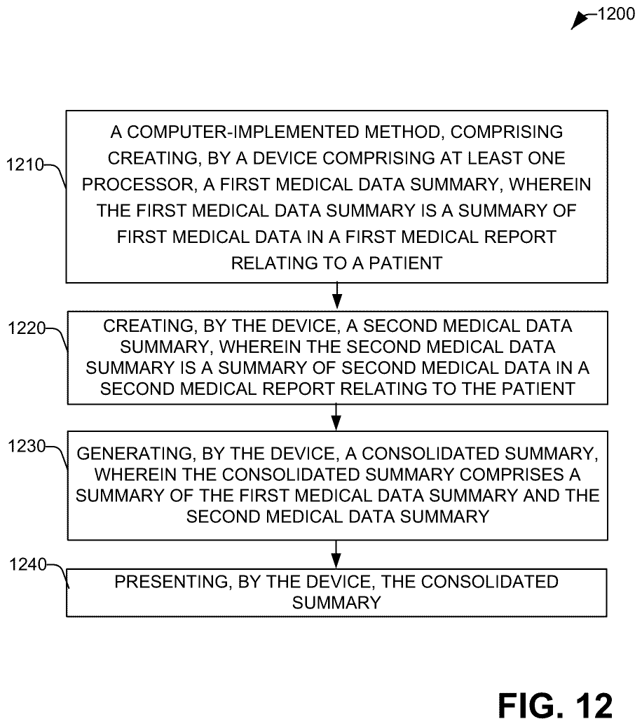

FIG. 12, via flowchart 1200, presents an example computer-implemented method for automatically and dynamically generating one or more consolidated summaries comprising and linked to summary reports and further to patient reports, in accordance with an embodiment.

At 1210, method 1200 can comprise creating, by a device (e.g., RIS 110) comprising at least one processor (e.g., processor 182A-n), a first medical data summary (e.g., first medical summary 117A), wherein the first medical data summary is a summary of first medical data in a first medical report (e.g., patient report 115A-n) relating to a patient (e.g., patient 102).

At 1220, method 1200 can further comprise creating, by the device, a second medical data summary (e.g., second medical summary 117B), wherein the second medical data summary is a summary of second medical data in a second medical report (e.g., patient report 115A-n) relating to the patient.

At 1230, method 1200 can further comprise generating, by the device, a consolidated summary (e.g., consolidated summary 118A), wherein the consolidated summary comprises a summary of the first medical data summary (e.g., first medical summary 117A) and the second medical data summary (e.g., second medical summary 117B).

At 1240, method 1200 can further comprise presenting (e.g., on HMI 186), by the device, the consolidated summary.

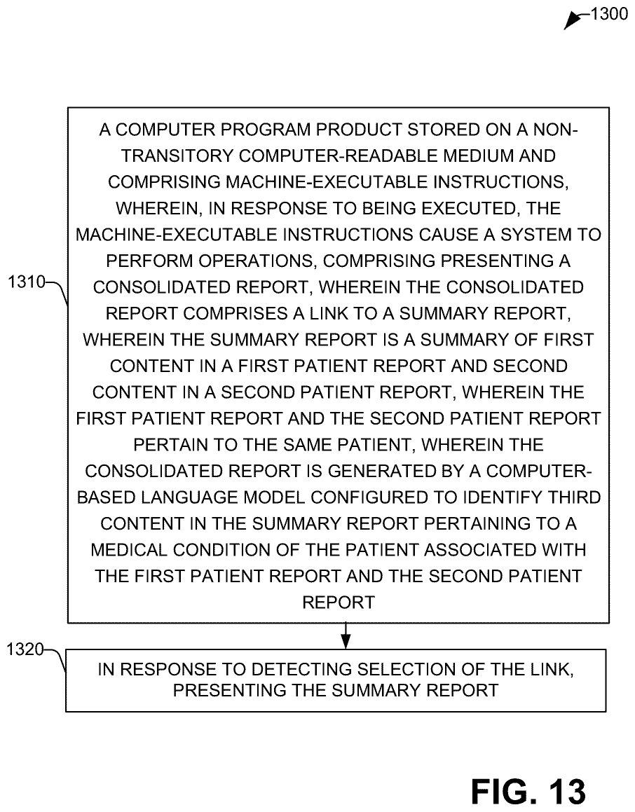

FIG. 13, via flowchart 1300, presents an example computer-implemented method for automatically and dynamically generating one or more consolidated summaries comprising and linked to summary reports and further to patient reports, in accordance with an embodiment.

At 1310, method 1300 can be performed with a computer program product stored on a non-transitory computer-readable medium (e.g., memory 184A-n) and comprising machine-executable instructions, wherein, in response to being executed (e.g., by processor 182A-n), the machine-executable instructions cause a system (e.g., RIS 110) to perform operations, comprising presenting a consolidated report (e.g., consolidated summary 118A), wherein the consolidated report comprises a link (e.g., link 113A-n) to a summary report (e.g., report summary 117A), wherein the summary report is a summary of first content in a first patient report (e.g., patient report 115A) and second content in a second patient report (e.g., patient report 115B), wherein the first patient report and the second patient report pertain to the same patient (e.g., patient 102), wherein the consolidated report is generated by a computer-based language model (e.g., process 127A-n) configured to identify third content in the summary report pertaining to a medical condition of the patient associated with the first patient report and the second patient report

At 1320, method 1300 can further comprise in response to detecting selection (e.g., by summary component 125) of the link, presenting the summary report (e.g., report summary 117A).

### Example Applications and Use

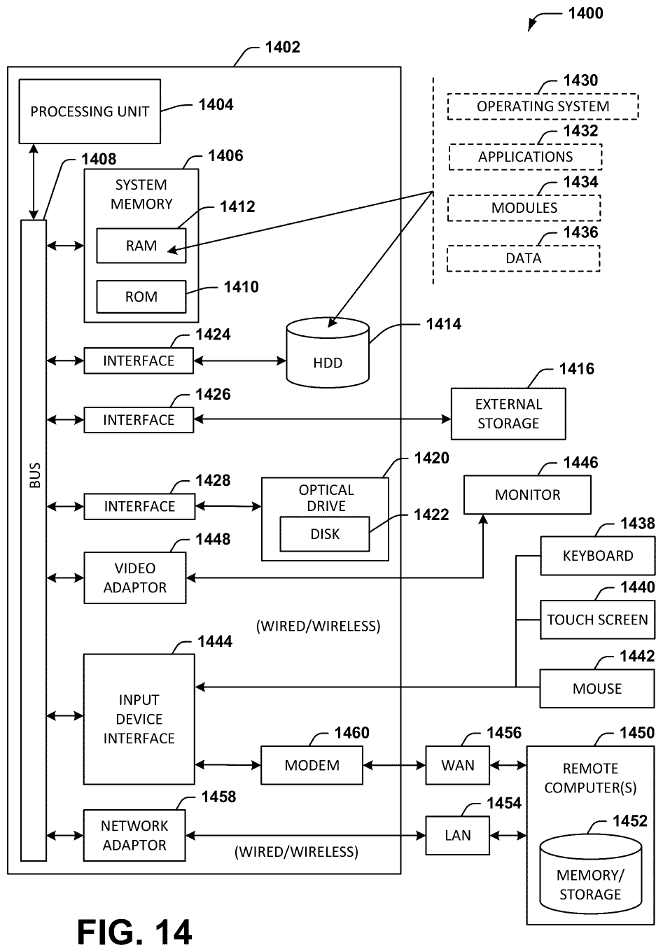

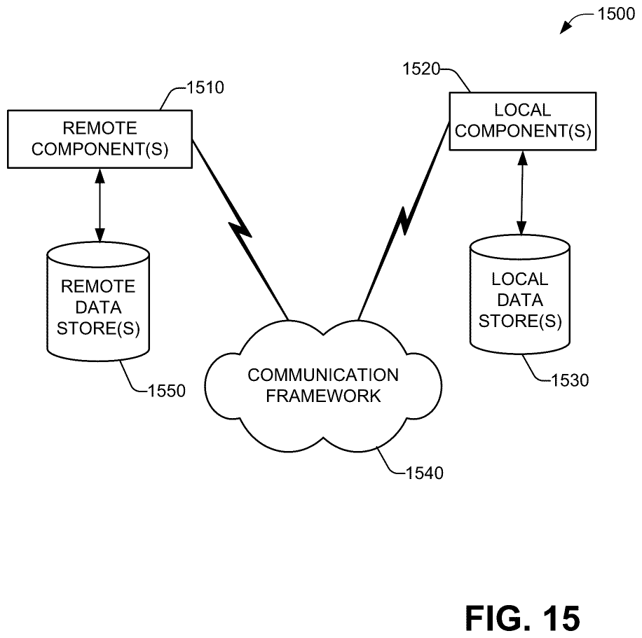

Turning next to FIGS. 14 and 15, a detailed description is provided of additional context for the one or more embodiments described herein with FIGS. 1A-13.

In order to provide additional context for various embodiments described herein, FIG. 14 and the following discussion are intended to provide a brief, general description of a suitable computing environment 1400 in which the various embodiments described herein can be implemented. While the embodiments have been described above in the general context of computer-executable instructions that can run on one or more computers, those skilled in the art will recognize that the embodiments can be also implemented in combination with other program modules and/or as a combination of hardware and software.

Generally, program modules include routines, programs, components, data structures, etc., that perform particular tasks or implement particular abstract data types. Moreover, those skilled in the art will appreciate that the methods can be practiced with other computer system configurations, including single-processor or multiprocessor computer systems, minicomputers, mainframe computers, IoT devices, distributed computing systems, as well as personal computers, hand-held computing devices, microprocessor-based or programmable consumer electronics, and the like, each of which can be operatively coupled to one or more associated devices.

The embodiments illustrated herein can be also practiced in distributed computing environments where certain tasks are performed by remote processing devices that are linked through a communications network. In a distributed computing environment, program modules can be located in both local and remote memory storage devices.

Computing devices typically include a variety of media, which can include computer-readable storage media, machine-readable storage media, and/or communications media, which two terms are used herein differently from one another as follows. Computer-readable storage media or machine-readable storage media can be any available storage media that can be accessed by the computer and includes both volatile and nonvolatile media, removable and non-removable media. By way of example, and not limitation, computer-readable storage media or machine-readable storage media can be implemented in connection with any method or technology for storage of information such as computer-readable or machine-readable instructions, program modules, structured data or unstructured data.

Computer-readable storage media can include, but are not limited to, random access memory (RAM), read only memory (ROM), electrically erasable programmable read only memory (EEPROM), flash memory or other memory technology, compact disk read only memory (CD-ROM), digital versatile disk (DVD), Blu-ray disc (BD) or other optical disk storage, magnetic cassettes, magnetic tape, magnetic disk storage or other magnetic storage devices, solid state drives or other solid state storage devices, or other tangible and/or non-transitory media which can be used to store desired information. In this regard, the terms “tangible” or “non-transitory” herein as applied to storage, memory or computer-readable media, are to be understood to exclude only propagating transitory signals per se as modifiers and do not relinquish rights to all standard storage, memory or computer-readable media that are not only propagating transitory signals per se.

Computer-readable storage media can be accessed by one or more local or remote computing devices, e.g., via access requests, queries or other data retrieval protocols, for a variety of operations with respect to the information stored by the medium.

Communications media typically embody computer-readable instructions, data structures, program modules or other structured or unstructured data in a data signal such as a modulated data signal, e.g., a carrier wave or other transport mechanism, and includes any information delivery or transport media. The term “modulated data signal” or signals refers to a signal that has one or more of its characteristics set or changed in such a manner as to encode information in one or more signals. By way of example, and not limitation, communication media include wired media, such as a wired network or direct-wired connection, and wireless media such as acoustic, RF, infrared and other wireless media.

With reference again to FIG. 14, the example environment 1400 for implementing various embodiments of the aspects described herein includes a computer 1402, the computer 1402 including a processing unit 1404, a system memory 1406 and a system bus 1408. The system bus 1408 couples system components including, but not limited to, the system memory 1406 to the processing unit 1404. The processing unit 1404 can be any of various commercially available processors and may include a cache memory. Dual microprocessors and other multi-processor architectures can also be employed as the processing unit 1404.

The system bus 1408 can be any of several types of bus structure that can further interconnect to a memory bus (with or without a memory controller), a peripheral bus, and a local bus using any of a variety of commercially available bus architectures. The system memory 1406 includes ROM 1410 and RAM 1412. A basic input/output system (BIOS) can be stored in a non-volatile memory such as ROM, erasable programmable read only memory (EPROM), EEPROM, which BIOS contains the basic routines that help to transfer information between elements within the computer 1402, such as during startup. The RAM 1412 can also include a high-speed RAM such as static RAM for caching data.

The computer 1402 further includes an internal hard disk drive (HDD) 1414 (e.g., EIDE, SATA), one or more external storage devices 1416 (e.g., a magnetic floppy disk drive (FDD) 1416, a memory stick or flash drive reader, a memory card reader, etc.) and an optical disk drive 1420 (e.g., which can read or write from a CD-ROM disc, a DVD, a BD, etc.). While the internal HDD 1414 is illustrated as located within the computer 1402, the internal HDD 1414 can also be configured for external use in a suitable chassis (not shown). Additionally, while not shown in environment 1400, a solid-state drive (SSD) could be used in addition to, or in place of, an HDD 1414. The HDD 1414, external storage device(s) 1416 and optical disk drive 1422 can be connected to the system bus 1408 by an HDD interface 1424, an external storage interface 1426 and an optical drive interface 1428, respectively. The interface 1424 for external drive implementations can include at least one or both of Universal Serial Bus (USB) and Institute of Electrical and Electronics Engineers (IEEE) 1394 interface technologies. Other external drive connection technologies are within contemplation of the embodiments described herein.

The drives and their associated computer-readable storage media provide nonvolatile storage of data, data structures, computer-executable instructions, and so forth. For the computer 1402, the drives and storage media accommodate the storage of any data in a suitable digital format. Although the description of computer-readable storage media above refers to respective types of storage devices, it should be appreciated by those skilled in the art that other types of storage media which are readable by a computer, whether presently existing or developed in the future, could also be used in the example operating environment, and further, that any such storage media can contain computer-executable instructions for performing the methods described herein.

A number of program modules can be stored in the drives and RAM 1412, including an operating system 1430, one or more application programs 1432, other program modules 1434 and program data 1436. All or portions of the operating system, applications, modules, and/or data can also be cached in the RAM 1412. The systems and methods described herein can be implemented utilizing various commercially available operating systems or combinations of operating systems.

Computer 1402 can optionally comprise emulation technologies. For example, a hypervisor (not shown) or other intermediary can emulate a hardware environment for operating system 1430, and the emulated hardware can optionally be different from the hardware illustrated in FIG. 14. In such an embodiment, operating system 1430 can comprise one virtual machine (VM) of multiple VMs hosted at computer 1402. Furthermore, operating system 1430 can provide runtime environments, such as the Java runtime environment or the .NET framework, for applications 1432. Runtime environments are consistent execution environments that allow applications 1432 to run on any operating system that includes the runtime environment. Similarly, operating system 1430 can support containers, and applications 1432 can be in the form of containers, which are lightweight, standalone, executable packages of software that include, e.g., code, runtime, system tools, system libraries and settings for an application.

Further, computer 1402 can comprise a security module, such as a trusted processing module (TPM). For instance with a TPM, boot components hash next in time boot components, and wait for a match of results to secured values, before loading a next boot component. This process can take place at any layer in the code execution stack of computer 1402, e.g., applied at the application execution level or at the operating system (OS) kernel level, thereby enabling security at any level of code execution.

A user can enter commands and information into the computer 1402 through one or more wired/wireless input devices, e.g., a keyboard 1438, a touch screen 1440, and a pointing device, such as a mouse 1442. Other input devices (not shown) can include a microphone, an infrared (IR) remote control, a radio frequency (RF) remote control, or other remote control, a joystick, a virtual reality controller and/or virtual reality headset, a game pad, a stylus pen, an image input device, e.g., camera(s), a gesture sensor input device, a vision movement sensor input device, an emotion or facial detection device, a biometric input device, e.g., fingerprint or iris scanner, or the like. These and other input devices are often connected to the processing unit 1404 through an input device interface 1444 that can be coupled to the system bus 1408, but can be connected by other interfaces, such as a parallel port, an IEEE 1394 serial port, a game port, a USB port, an IR interface, a BLUETOOTH® interface, etc.

A monitor 1446 or other type of display device can be also connected to the system bus 1408 via an interface, such as a video adapter 1448. In addition to the monitor 1446, a computer typically includes other peripheral output devices (not shown), such as speakers, printers, etc.

The computer 1402 can operate in a networked environment using logical connections via wired and/or wireless communications to one or more remote computers, such as a remote computer(s) 1450. The remote computer(s) 1450 can be a workstation, a server computer, a router, a personal computer, portable computer, microprocessor-based entertainment appliance, a peer device or other common network node, and typically includes many or all of the elements described relative to the computer 1402, although, for purposes of brevity, only a memory/storage device 1452 is illustrated. The logical connections depicted include wired/wireless connectivity to a local area network (LAN) 1454 and/or larger networks, e.g., a wide area network (WAN) 1456. Such LAN and WAN networking environments are commonplace in offices and companies, and facilitate enterprise-wide computer networks, such as intranets, all of which can connect to a global communications network, e.g., the internet.

When used in a LAN networking environment, the computer 1402 can be connected to the local network 1454 through a wired and/or wireless communication network interface or adapter 1458. The adapter 1458 can facilitate wired or wireless communication to the LAN 1454, which can also include a wireless access point (AP) disposed thereon for communicating with the adapter 1458 in a wireless mode.

When used in a WAN networking environment, the computer 1402 can include a modem 1460 or can be connected to a communications server on the WAN 1456 via other means for establishing communications over the WAN 1456, such as by way of the internet. The modem 1460, which can be internal or external and a wired or wireless device, can be connected to the system bus 1408 via the input device interface 1444. In a networked environment, program modules depicted relative to the computer 1402 or portions thereof, can be stored in the remote memory/storage device 1452. It will be appreciated that the network connections shown are example and other means of establishing a communications link between the computers can be used.

When used in either a LAN or WAN networking environment, the computer 1402 can access cloud storage systems or other network-based storage systems in addition to, or in place of, external storage devices 1416 as described above. Generally, a connection between the computer 1402 and a cloud storage system can be established over a LAN 1454 or WAN 1456 e.g., by the adapter 1458 or modem 1460, respectively. Upon connecting the computer 1402 to an associated cloud storage system, the external storage interface 1426 can, with the aid of the adapter 1458 and/or modem 1460, manage storage provided by the cloud storage system as it would other types of external storage. For instance, the external storage interface 1426 can be configured to provide access to cloud storage sources as if those sources were physically connected to the computer 1402.

The computer 1402 can be operable to communicate with any wireless devices or entities operatively disposed in wireless communication, e.g., a printer, scanner, desktop and/or portable computer, portable data assistant, communications satellite, any piece of equipment or location associated with a wirelessly detectable tag (e.g., a kiosk, news stand, store shelf, etc.), and telephone. This can include Wireless Fidelity (Wi-Fi) and BLUETOOTH® wireless technologies. Thus, the communication can be a predefined structure as with a conventional network or simply an ad hoc communication between at least two devices.

The above description includes non-limiting examples of the various embodiments. It is, of course, not possible to describe every conceivable combination of components or methodologies for purposes of describing the disclosed subject matter, and one skilled in the art may recognize that further combinations and permutations of the various embodiments are possible. The disclosed subject matter is intended to embrace all such alterations, modifications, and variations that fall within the spirit and scope of the appended claims.

Referring now to details of one or more elements illustrated at FIG. 15, an illustrative cloud computing environment 1500 is depicted. FIG. 15 is a schematic block diagram of a computing environment 1500 with which the disclosed subject matter can interact. The system 1500 comprises one or more remote component(s) 1510. The remote component(s) 1510 can be hardware and/or software (e.g., threads, processes, computing devices). In some embodiments, remote component(s) 1510 can be a distributed computer system, connected to a local automatic scaling component and/or programs that use the resources of a distributed computer system, via communication framework 1540. Communication framework 1540 can comprise wired network devices, wireless network devices, mobile devices, wearable devices, radio access network devices, gateway devices, femtocell devices, servers, etc.

The system 1500 also comprises one or more local component(s) 1520. The local component(s) 1520 can be hardware and/or software (e.g., threads, processes, computing devices). In some embodiments, local component(s) 1520 can comprise an automatic scaling component and/or programs that communicate/use the remote resources 1510 and 1520, etc., connected to a remotely located distributed computing system via communication framework 1540.

One possible communication between a remote component(s) 1510 and a local component(s) 1520 can be in the form of a data packet adapted to be transmitted between two or more computer processes. Another possible communication between a remote component(s) 1510 and a local component(s) 1520 can be in the form of circuit-switched data adapted to be transmitted between two or more computer processes in radio time slots. The system 1500 comprises a communication framework 1540 that can be employed to facilitate communications between the remote component(s) 1510 and the local component(s) 1520, and can comprise an air interface, e.g., Uu interface of a UMTS network, via a long-term evolution (LTE) network, etc. Remote component(s) 1510 can be operably connected to one or more remote data store(s) 1550, such as a hard drive, solid state drive, SIM card, device memory, etc., that can be employed to store information on the remote component(s) 1510 side of communication framework 1540. Similarly, local component(s) 1520 can be operably connected to one or more local data store(s) 1530, that can be employed to store information on the local component(s) 1520 side of communication framework 1540.

With regard to the various functions performed by the above described components, devices, circuits, systems, etc., the terms (including a reference to a “means”) used to describe such components are intended to also include, unless otherwise indicated, any structure(s) which performs the specified function of the described component (e.g., a functional equivalent), even if not structurally equivalent to the disclosed structure. In addition, while a particular feature of the disclosed subject matter may have been disclosed with respect to only one of several implementations, such feature may be combined with one or more other features of the other implementations as may be desired and advantageous for any given or particular application.

The terms “exemplary” and/or “demonstrative” as used herein are intended to mean serving as an example, instance, or illustration. For the avoidance of doubt, the subject matter disclosed herein is not limited by such examples. In addition, any aspect or design described herein as “exemplary” and/or “demonstrative” is not necessarily to be construed as preferred or advantageous over other aspects or designs, nor is it meant to preclude equivalent structures and techniques known to one skilled in the art. Furthermore, to the extent that the terms “includes,” “has,” “contains,” and other similar words are used in either the detailed description or the claims, such terms are intended to be inclusive—in a manner similar to the term “comprising” as an open transition word—without precluding any additional or other elements.

The term “or” as used herein is intended to mean an inclusive “or” rather than an exclusive “or.” For example, the phrase “A or B” is intended to include instances of A, B, and both A and B. Additionally, the articles “a” and “an” as used in this application and the appended claims should generally be construed to mean “one or more” unless either otherwise specified or clear from the context to be directed to a singular form.

The term “set” as employed herein excludes the empty set, i.e., the set with no elements therein. Thus, a “set” in the subject disclosure includes one or more elements or entities. Likewise, the term “group” as utilized herein refers to a collection of one or more entities.

The terms “first,” “second,” “third,” and so forth, as used in the claims, unless otherwise clear by context, is for clarity only and doesn't otherwise indicate or imply any order in time. For instance, “a first determination,” “a second determination,” and “a third determination,” does not indicate or imply that the first determination is to be made before the second determination, or vice versa, etc.

As used in this disclosure, in some embodiments, the terms “component,” “system” and the like are intended to refer to, or comprise, a computer-related entity or an entity related to an operational apparatus with one or more specific functionalities, wherein the entity can be either hardware, a combination of hardware and software, software, or software in execution. As an example, a component can be, but is not limited to being, a process running on a processor, a processor, an object, an executable, a thread of execution, computer-executable instructions, a program, and/or a computer. By way of illustration and not limitation, both an application running on a server and the server can be a component.

One or more components can reside within a process and/or thread of execution and a component can be localized on one computer and/or distributed between two or more computers. In addition, these components can execute from various computer readable media having various data structures stored thereon. The components can communicate via local and/or remote processes such as in accordance with a signal having one or more data packets (e.g., data from one component interacting with another component in a local system, distributed system, and/or across a network such as the internet with other systems via the signal). As another example, a component can be an apparatus with specific functionality provided by mechanical parts operated by electric or electronic circuitry, which is operated by a software application or firmware application executed by a processor, wherein the processor can be internal or external to the apparatus and executes at least a part of the software or firmware application. As yet another example, a component can be an apparatus that provides specific functionality through electronic components without mechanical parts, the electronic components can comprise a processor therein to execute software or firmware that confers at least in part the functionality of the electronic components. While various components have been illustrated as separate components, it will be appreciated that multiple components can be implemented as a single component, or a single component can be implemented as multiple components, without departing from example embodiments.

The term “facilitate” as used herein is in the context of a system, device or component “facilitating” one or more actions or operations, in respect of the nature of complex computing environments in which multiple components and/or multiple devices can be involved in some computing operations. Non-limiting examples of actions that may or may not involve multiple components and/or multiple devices comprise transmitting or receiving data, establishing a connection between devices, determining intermediate results toward obtaining a result, etc. In this regard, a computing device or component can facilitate an operation by playing any part in accomplishing the operation. When operations of a component are described herein, it is thus to be understood that where the operations are described as facilitated by the component, the operations can be optionally completed with the cooperation of one or more other computing devices or components, such as, but not limited to, sensors, antennae, audio and/or visual output devices, other devices, etc.

Further, the various embodiments can be implemented as a method, apparatus or article of manufacture using standard programming and/or engineering techniques to produce software, firmware, hardware, or any combination thereof to control a computer to implement the disclosed subject matter. The term “article of manufacture” as used herein is intended to encompass a computer program accessible from any computer-readable (or machine-readable) device or computer-readable (or machine-readable) storage/communications media. For example, computer readable storage media can comprise, but are not limited to, magnetic storage devices (e.g., hard disk, floppy disk, magnetic strips), optical disks (e.g., compact disk (CD), digital versatile disk (DVD)), smart cards, and flash memory devices (e.g., card, stick, key drive). Of course, those skilled in the art will recognize many modifications can be made to this configuration without departing from the scope or spirit of the various embodiments.

Moreover, terms such as “mobile device equipment,” “mobile station,” “mobile,” “subscriber station,” “access terminal,” “terminal,” “handset,” “communication device,” “mobile device” (and/or terms representing similar terminology) can refer to a wireless device utilized by a subscriber or mobile device of a wireless communication service to receive or convey data, control, voice, video, sound, gaming or substantially any data-stream or signaling-stream. The foregoing terms are utilized interchangeably herein and with reference to the related drawings. Likewise, the terms “access point (AP),” “Base Station (BS),” “BS transceiver,” “BS device,” “cell site,” “cell site device,” “gNode B (gNB),” “evolved Node B (eNode B, eNB),” “home Node B (HNB)” and the like, refer to wireless network components or appliances that transmit and/or receive data, control, voice, video, sound, gaming or substantially any data-stream or signaling-stream from one or more subscriber stations. Data and signaling streams can be packetized or frame-based flows.

Furthermore, the terms “device,” “communication device,” “mobile device,” “subscriber,” “client entity,” “consumer,” “client entity,” “entity” and the like are employed interchangeably throughout, unless context warrants particular distinctions among the terms. It should be appreciated that such terms can refer to human entities or automated components supported through artificial intelligence (e.g., a capacity to make inference based on complex mathematical formalisms), which can provide simulated vision, sound recognition and so forth.

It should be noted that although various aspects and embodiments are described herein in the context of 5G or other next generation networks, the disclosed aspects are not limited to a 5G implementation, and can be applied in other network next generation implementations, such as sixth generation (6G), or other wireless systems. In this regard, aspects or features of the disclosed embodiments can be exploited in substantially any wireless communication technology. Such wireless communication technologies can include universal mobile telecommunications system (UMTS), global system for mobile communication (GSM), code division multiple access (CDMA), wideband CDMA (WCMDA), CDMA2000, time division multiple access (TDMA), frequency division multiple access (FDMA), multi-carrier CDMA (MC-CDMA), single-carrier CDMA (SC-CDMA), single-carrier FDMA (SC-FDMA), orthogonal frequency division multiplexing (OFDM), discrete Fourier transform spread OFDM (DFT-spread OFDM), filter bank based multi-carrier (FBMC), zero tail DFT-spread-OFDM (ZT DFT-s-OFDM), generalized frequency division multiplexing (GFDM), fixed mobile convergence (FMC), universal fixed mobile convergence (UFMC), unique word OFDM (UW-OFDM), unique word DFT-spread OFDM (UW DFT-Spread-OFDM), cyclic prefix OFDM (CP-OFDM), resource-block-filtered OFDM, wireless fidelity (Wi-Fi), worldwide interoperability for microwave access (WiMAX), wireless local area network (WLAN), general packet radio service (GPRS), enhanced GPRS, third generation partnership project (3GPP), long term evolution (LTE), 5G, third generation partnership project 2 (3GPP2), ultra-mobile broadband (UMB), high speed packet access (HSPA), evolved high speed packet access (HSPA+), high-speed downlink packet access (HSDPA), high-speed uplink packet access (HSUPA), Zigbee, or another institute of electrical and electronics engineers (IEEE) 802.12 technology.

It is to be understood that when an element is referred to as being “coupled” to another element, it can describe one or more different types of coupling including, but not limited to, chemical coupling, communicative coupling, electrical coupling, electromagnetic coupling, operative coupling, optical coupling, physical coupling, thermal coupling, and/or another type of coupling. Likewise, it is to be understood that when an element is referred to as being “connected” to another element, it can describe one or more different types of connecting including, but not limited to, electrical connecting, electromagnetic connecting, operative connecting, optical connecting, physical connecting, thermal connecting, and/or another type of connecting.

The description of illustrated embodiments of the subject disclosure as provided herein, including what is described in the Abstract, is not intended to be exhaustive or to limit the disclosed embodiments to the precise forms disclosed. While specific embodiments and examples are described herein for illustrative purposes, various modifications are possible that are considered within the scope of such embodiments and examples, as one skilled in the art can recognize. In this regard, while the subject matter has been described herein in connection with various embodiments and corresponding drawings, where applicable, it is to be understood that other similar embodiments can be used or modifications and additions can be made to the described embodiments for performing the same, similar, alternative, or substitute function of the disclosed subject matter without deviating therefrom. Therefore, the disclosed subject matter should not be limited to any single embodiment described herein, but rather should be construed in breadth and scope in accordance with the appended claims below.

What is claimed is:

1. A system, comprising:
at least one processor; and
at least one memory coupled to the at least one processor and having instructions stored thereon, wherein, in response to the at least one processor executing the instructions, the instructions facilitate performance of operations, comprising:
generating a first report summary in a collection of report summaries, wherein the first report summary comprises a first summary of first patient medical data in a first patient report pertaining to a patient, and the first report summary is generated in accordance with a first prompt applied to a large language model operating on the first patient report;
generating a second report summary in the collection of report summaries, wherein the second report summary comprises a first summary of second patient medical data in a second patient report pertaining to the patient, and the second report summary is generated in accordance with the first prompt applied to the large language model operating on the first patient report;
generating a consolidated summary of a patient's medical history pertaining to the patient, wherein the consolidated summary is generated from the first report summary and the second report summary, and the consolidated report is generated in accordance with a second prompt applied to the large language model operating on the first report summary and the second report summary; and
presenting the consolidated summary for review.

2. The system of claim 1, wherein the operations further comprise:
detecting a selection from the consolidated summary;
determining patient medical data corresponding to the selection; and
presenting the patient medical data pertaining to the selection.

3. The system of claim 1, wherein the consolidated summary is presented on a human-machine interface (HMI), and at least one of the first patient medical data pertaining to the selection is co-presented with the consolidated summary on the HMI or the second patient medical data pertaining to the selection is co-presented with the consolidated summary on the HMI.

4. The system of claim 1, wherein the first patient medical data and the second patient medical data comprises at least one of an image, medical information pertaining to the patient, patient personal information, or medical research information pertinent to the patient.

5. The system of claim 1, wherein the at least one medical report comprises at least one of a radiology report, a oncology report, a cardiology report, a neurology report, a patient intervention report, an intensive care report, a general practitioner report, a report pertaining to the patient, a note pertaining to the patient, a report in an electronic medical record (EMR) system pertaining to the patient, a report in a non-EMR system pertaining to the patient, or a medical specialty report.

6. The system of claim 1, wherein the first report summary comprises a summary of patient data included in at least one medical report.

7. The system of claim 1, wherein the first report summary comprises a summary of at least one of patient activity or patient testing performed between a first date and a second date.

8. The system of claim 1, wherein the consolidated summary further includes one or more links, wherein a first link is a hyperlink to a first medical report in the at least one medical report, and the operations further comprising:
detecting selection of the first link; and
presenting the first medical report.

9. The system of claim 1, wherein the operations further comprise:
identifying first content in original patient reports pertaining to second content in a query received at the system, wherein identification of the first content is based on vector similarity between the first content in the original patient reports and the second content of the query, wherein the original patient reports include the first patient report and the second patient report;
generate a query answer using the large language model based on the first content and the second content; and
presenting the query answer for review.

10. The system of claim 1, wherein the at least one medical report comprises a plurality of medical reports, and the consolidated summary is a first consolidated summary comprising oncology data generated from a first report summary, wherein the first report summary is generated from one or more oncology reports included in the plurality of medical reports, and a second consolidated summary comprises radiology data generated from a third report summary, wherein the third report summary is generated from one or more radiology reports included in the plurality of medical reports.

11. A computer-implemented method, comprising:
creating, by a device comprising at least one processor, a first medical data summary, wherein the first medical data summary is a summary of first medical data in a first medical report relating to a patient;
creating, by the device, a second medical data summary, wherein the second medical data summary is a summary of second medical data in a second medical report relating to the patient;
generating, by the device, a consolidated summary, wherein the consolidated summary comprises a summary of the first medical data summary and the second medical data summary; and
presenting, by the device, the consolidated summary.

12. The computer-implemented method of claim 11, further comprising:
generating, by the device, in the consolidated summary a hyperlink, wherein the hyperlink is a digital reference to the first medical data summary;
detecting, by the device, selection of the hyperlink; and
presenting, by the device, the first medical data summary in conjunction with the consolidated summary.

13. The computer-implemented method of claim 11 further comprising:
generating, by the device, in the consolidated summary a first hyperlink, wherein the first hyperlink is a first digital reference to the first medical data summary;
generating, by the device, in the consolidated summary a second hyperlink, wherein the second hyperlink is a second digital reference to the second medical data summary;
detecting, by the device, selection of the of the first hyperlink or the second hyperlink; and
in response to detecting selection of the first hyperlink, presenting, by the device, the first medical data summary; and
in response to detecting selection of the second hyperlink, presenting, by the device, the second medical data summary.

14. The computer-implemented method of claim 11, further comprising:
receiving, by the device, an input comprising a question regarding information presented in the consolidated summary;
parsing, by the device, the information in the consolidated summary to generate an answer to the question; and
presenting, by the device, the answer.

15. The computer-implemented method of claim 14, further comprising, prior to parsing the information in the consolidated summary, applying a first prompt to the question, wherein the first prompt applies a filter to the question to further refine the question for application in parsing the information in the consolidated summary.

16. The computer-implemented method of claim 15, further comprising,
capturing, by the device, interaction with the answer; and
generating, by the device, a second prompt, wherein the second prompt comprises the first prompt adjusted in accordance with the interaction.

17. A computer program product stored on a non-transitory computer-readable medium and comprising machine-executable instructions, wherein, in response to being executed, the machine-executable instructions cause a system to perform operations, comprising:
presenting a consolidated report, wherein the consolidated report comprises a link to a summary report, wherein the summary report is a summary of first content in a first patient report and second content in a second patient report, wherein the first patient report and the second patient report pertain to the same patient, wherein the consolidated report is generated by a computer-based language model configured to identify third content in the summary report pertaining to a medical condition of the patient associated with the first patient report and the second patient report; and
in response to detecting selection of the link, presenting the summary report.

18. The computer program product according to claim 17, wherein the link to the first summary report is a first link, and the first summary report further includes a second link to the first patient report and a third link to the second patient report, where operations further comprising:
in response to detecting selection of the second link, presenting the first patient report; and
in response to detecting selection of the third link, presenting the second patient report.

19. The computer program product according to claim 17, the operations further comprising:
receiving a query having fourth content, wherein the query pertains to fifth content presented in the consolidated report, wherein the fifth content is vectorized content;
vectorizing the fourth content of the query;
identifying the fifth content based on vector similarity between the vectorized fifth content and the vectorized fourth content; and
presenting the fifth content.

20. The computer program product according to claim 19, wherein the computer-based language model is a first computer-based model and the consolidated report is a first consolidated report, wherein the operations further comprising:
receiving a modification to the fifth content;
updating the consolidated report in accordance with the modification;
generating a second computer-based language model, wherein the second
computer-based language model is generated in accordance with the modification to the consolidated report; and
subsequently applying the second computer-based language model to the summary report to generate a second consolidated report.
